(19) United States
(12) Patent Application Publication
(10) Pub. No.: US 2026/0123837 A1
(43) Pub. Date: May 7, 2026

(54) ULTRALOW POWER MEMORY FOR IMPLANTED DEVICE

(71) Applicant: Iota Biosciences, Inc., Alameda, CA (US)

(72) Inventors: Dwight David GRIFFIN, San Jose, CA (US); Yasha KARIMI, San Jose, CA (US); Gaurav MITTAL, Pleasanton, CA (US); Pujitha WEERAKOON, Moraga, CA (US); Maruthi MUKKANNAIAH, Fremont, CA (US)

(21) Appl. No.: 19/118,349

(22) PCT Filed: Oct. 18, 2023
(86) PCT No.: PCT/US2023/077219

§ 371 Date: Apr. 3, 2025

Related U.S. Application Data
(60) Provisional application No. 63/417,639, filed on Oct. 19, 2022.

(51) Int. Cl. A61B 5/00; A61B 5/03; A61B 5/28

(52) U.S. Cl. CPC A61B 5/0031; A61B 5/031; A61B 5/28; A61B 2560/0219; A61B 2562/0247

(57) ABSTRACT

An implantable device may comprise a power management unit configured to receive power from an external power source, one or more sensors, and a memory configured to store data detected by the one or more sensors. The memory may include a first binary storage element electrically coupled to the sensors and to a main power supply line coupled to the power management unit, and a second binary storage element coupled to the first binary data memory storage element and to a retention power supply line connected to the power supply. The device may include a digital circuit configured to determine that the power management unit is not receiving power from the external power source, detect that a predetermined condition has been met, store data collected by the sensors in the second binary data memory storage element, and disconnect the main power supply line from the power supply.

### CROSS-REFERENCE TO RELATED APPLICATIONS

This application claims the priority benefit of U.S. patent application Ser. No. 63/417,639, filed Oct. 19, 2022, the entire contents of which are incorporated herein by reference for all purposes.

### FIELD

The present invention relates to memory systems for implantable devices, in particular for implantable devices configured to detect a physiological signal in a subject.

### BACKGROUND

Implantable devices for monitoring physiological signals in a subject can collect data that provides a better understanding of health and disease prognosis. For example, implantable devices for blood sugar monitoring may be used to monitor the health of a diabetic patient, and implantable devices for monitoring blood oxygenation levels may be used to monitor compartment syndrome, cancer, or organ transplants.

Due to their size, certain implantable devices may rely primarily on energy that is harvested or transferred from a source outside of the device to operate. While implantable devices may have the capacity to store energy provided by the external energy source, this capacity may be extremely limited. When the external source of energy is removed, implantable devices may need to perform operations and retain essential amounts of data without depleting the energy stored on the device. Conventional reprogrammable non-volatile memory elements (e.g., Flash), which retain data when power is lost, require large amounts of energy to program the data. Furthermore, conventional reprogrammable non-volatile memory elements may be too large for applications where only small amounts of data need to be retained. Conventional volatile memory elements (e.g., SRAM) require large amounts of energy to store data and may completely deplete the energy stored on the device when energy is not being provided by the external source.

### SUMMARY

The implantable devices described herein have a memory element that can retain small amounts of data using very low amounts of power while the rest of the device is in a low power “sleep” mode. In some embodiments, the memory element has at least two separate binary data memory storage elements, each of which may be independently coupled to a power supply of the device. When the device is not receiving energy from an external power source, a primary binary data memory storage element, which may store data collected by the device when the device has sufficient power available, may be disconnected from the power supply in order to preserve the stored energy for an extended period of time. A secondary “retention” binary data memory storage element may remain connected to the power supply and may store a small amount of data that is essential to the device's continued operation.

An implantable device provided herein may include an energy storage configured to receive power from an external power source, one or more sensors configured to measure physiological signals, a memory configured to store physiological signal data measured by the one or more sensor, and a digital circuit. The memory may include a first binary data memory storage element electrically coupled to the one or more sensors and a main power supply line electrically coupled to the energy storage and a second binary data memory storage element electrically coupled to the first binary data memory storage element and a retention power supply line electrically coupled to the energy storage. The first binary data memory storage element may comprise a flip-flop circuit and the second binary data memory storage element may comprise a retention latch circuit. The digital circuit may be configured to determine that the power management unit is not receiving power from the external power source, detect that a predetermined condition has been met, store an essential portion of the physiological signal data that has been measured by the one or more sensors in the second binary data memory storage element, and disconnect the main power supply line from the energy storage. The essential portion of the physiological signal data that is stored in the second binary data memory storage element comprises data associated with a most recently performed measurement.

The main power supply line may be configured to transmit a first amount of power from the energy storage to the first binary data memory storage element and the retention power supply line may be configured to transmit a second amount of power from the energy storage to the second binary data memory storage element. The second amount of power may be less than the first amount of power. In some embodiments, the second amount of power is less than or equal to 100 picowatts.

The one or more sensors can include a pressure sensor. The pressure sensor may be configured to measure an intraocular pressure. Additionally or alternatively, the one or more sensors can include one or more electrodes configured to detect an electrophysiological pulse, a sensor configured to detect a concentration of an analyte, sensor configured to detect a pH, sensor configured to detect a temperature, sensor configured to detect an evoked action potential in a brain, sensor configured to detect a local field potential in a brain, or combinations thereof.

The device may be configured to be fully implantable and may be configured to be implanted in or attached to a tissue or organ. For example, the device may be configured to be implanted in an eye, on or in a central nervous tissue, on or in a brain, or on a peripheral nerve (e.g., a splenic nerve).

The energy storage may be configured to receive power wirelessly from the external power source. For example, the energy storage may be configured to receive power from ultrasonic waves produced by the external power source or from radio frequency waves produced by the external power source. Alternatively, the energy storage may be configured to receive power from the external power source via induction or via a capacitive link. The energy storage can also be configured to receive power from vibrations produced by the external power source using a vibrational transducer. In some embodiments, the energy storage comprises a capacitor; in some embodiments, the energy storage comprises a battery. In some embodiments, detecting that the predetermined condition has been met involves determining that a threshold time period from the time that the energy storage last received power from the external device has been exceeded. In other embodiments, detecting that the predetermined condition has been met involves determining that a total power level of the energy storage has dropped below a threshold power level.

Methods for collecting and storing data using an implantable device are also provided. The implantable device may comprise an energy storage configured to receive power from an external power source, one or more sensors configured to measure physiological signals, a memory configured to store physiological signal data measured by the one or more sensors, and a digital circuit. The memory can include a first binary data memory storage element electrically coupled to the one or more sensors and a main power supply line electrically coupled to the energy storage, and a second binary data memory storage element electrically coupled to the first binary data memory storage element and a retention power supply line electrically coupled to the energy storage. The first binary data memory storage element may comprise a flip-flop circuit and the second binary data memory storage element may comprise a retention latch circuit. A method for collecting and storing data using the implantable device may involve determining that the energy storage is no longer receiving power from the external power source, detecting that a predetermined condition has been met, storing an essential portion of the physiological signal data that has been measured by the one or more sensors in the second binary data memory storage element, and disconnecting the main power supply line from the energy storage. The essential portion of the physiological signal data that is stored in the second binary data memory storage element may include data associated with a most recently performed measurement.

The main power supply line may be configured to transmit a first amount of power from the energy storage to the first binary data memory storage element and the retention power supply line may be configured to transmit a second amount of power from the energy storage to the second binary data memory storage element. The second amount of power may be less than the first amount of power. In some embodiments, the second amount of power is less than or equal to 100 picowatts.

The one or more sensors can include a pressure sensor. The pressure sensor may be configured to measure an intraocular pressure. Additionally or alternatively, the one or more sensors can include one or more electrodes configured to detect an electrophysiological pulse, a sensor configured to detect a concentration of an analyte, sensor configured to detect a pH, sensor configured to detect a temperature, sensor configured to detect an evoked action potential in a brain, sensor configured to detect a local field potential in a brain, or combinations thereof.

The device may be configured to be fully implantable and may be configured to be implanted in or attached to a tissue or organ. For example, the device may be configured to be implanted in an eye, on or in a central nervous tissue, on or in a brain, or on a peripheral nerve (e.g., a splenic nerve).

The energy storage may be configured to receive power wirelessly from the external power source. For example, the energy storage may be configured to receive power from ultrasonic waves produced by the external power source or from radio frequency waves produced by the external power source. Alternatively, the energy storage may be configured to receive power from the external power source via induction or via a capacitive link. The energy storage can also be configured to receive power from vibrations produced by the external power source using a vibrational transducer. In some embodiments, the energy storage comprises a capacitor; in some embodiments, the energy storage comprises a battery. In some embodiments, detecting that the predetermined condition has been met involves determining that a threshold time period from the time that the energy storage last received power from the external device has been exceeded. In other embodiments, detecting that the predetermined condition has been met involves determining that a total power level of the energy storage has dropped below a threshold power level.

Non-transitory computer readable storage media containing instructions for collecting and storing data in an implantable device are also provided. The implantable device may comprise an energy storage configured to receive power from an external power source, one or more sensors configured to measure physiological signals, and a memory configured to store physiological signal data measured by the one or more sensors. The memory may include a first binary data memory storage element electrically coupled to the one or more sensors and a main power supply line electrically coupled to the energy storage, and a second binary data memory storage element electrically coupled to the first binary data memory storage element and a retention power supply line electrically coupled to the energy storage. The first binary data memory storage element may comprise a flip-flop circuit and the second binary data memory storage element may comprise a retention latch circuit. When executed by a digital circuit of an electronic device, instructions contained in a non-transitory computer readable storage medium provided herein may cause the electronic device to determine that the energy storage is not receiving power from the external power source, detect that a predetermined condition has been met; store an essential portion of the physiological signal data that has been measured by the one or more sensors in the second binary data memory storage element, and disconnect the main power supply line from the energy storage. The essential portion of the data that is stored in the second binary data memory storage element may include data associated with a most recently performed measurement.

The main power supply line may be configured to transmit a first amount of power from the energy storage to the first binary data memory storage element and the retention power supply line may be configured to transmit a second amount of power from the energy storage to the second binary data memory storage element. The second amount of power may be less than the first amount of power. In some embodiments, the second amount of power is less than or equal to 100 picowatts.

The one or more sensors can include a pressure sensor. The pressure sensor may be configured to measure an intraocular pressure. Additionally or alternatively, the one or more sensors can include one or more electrodes configured to detect an electrophysiological pulse, a sensor configured to detect a concentration of an analyte, sensor configured to detect a pH, sensor configured to detect a temperature, sensor configured to detect an evoked action potential in a brain, sensor configured to detect a local field potential in a brain, or combinations thereof.

The device may be configured to be fully implantable and may be configured to be implanted in or attached to a tissue or organ. For example, the device may be configured to be implanted in an eye, on or in a central nervous tissue, on or in a brain, or on a peripheral nerve (e.g., a splenic nerve).

The energy storage may be configured to receive power wirelessly from the external power source. For example, the energy storage may be configured to receive power from ultrasonic waves produced by the external power source or from radio frequency waves produced by the external power source. Alternatively, the energy storage may be configured to receive power from the external power source via induction or via a capacitive link. The energy storage can also be configured to receive power from vibrations produced by the external power source using a vibrational transducer. In some embodiments, the energy storage comprises a capacitor; in some embodiments, the energy storage comprises a battery. In some embodiments, detecting that the predetermined condition has been met involves determining that a threshold time period from the time that the energy storage last received power from the external device has been exceeded. In other embodiments, detecting that the predetermined condition has been met involves determining that a total power level of the energy storage has dropped below a threshold power level.

### BRIEF DESCRIPTION OF THE FIGURES

Various aspects of the disclosed methods and systems are set forth with particularity in the appended claims. A better understanding of the features and advantages of the disclosed methods and systems will be obtained by reference to the detailed description of illustrative embodiments and the accompanying drawings.

FIG. 1 shows a schematic of an exemplary system that includes an implantable device having an ultralow powered memory, according to some embodiments. The exemplary system is configured for measuring intraocular pressure.

FIG. 2 shows an exemplary implantable device comprising an ultralow power memory according to some embodiments of the present disclosure.

FIG. 3 shows an exemplary memory for an implantable device according to some embodiments of the present disclosure.

FIG. 4A shows an exemplary interrogator for providing energy to and communicating with an implantable device, according to some embodiments.

FIG. 4B shows an exemplary schematic of an exemplary interrogator for providing energy to and communicating with an ocular implantable device for measuring intraocular pressure, according to some embodiments.

FIG. 5 shows an exemplary interrogator in communication with an exemplary implantable device, according to some embodiments.

FIG. 6A shows an exemplary method for storing data using an implantable device according to some embodiments of the present disclosure.

FIG. 6B shows an exemplary method for detecting that an exemplary predetermined condition that may be associated with a power level in an implantable device has been met, according to some embodiments of the present disclosure.

FIG. 6C shows an exemplary method for detecting that an exemplary predetermined condition that may be associated with a power level in an implantable device has been met, according to some embodiments of the present disclosure.

FIG. 7 shows an exemplary method for retrieving data from an implantable device that was recently in a low power “sleep” mode.

FIG. 8 shows an exemplary method for using an intraocular implant to measure an intraocular pressure, store intraocular pressure data when the implant is in a low power “sleep” mode, and transmit the intraocular pressure data to an interrogator.

FIG. 9A shows an exemplary implementation of a first binary data memory storage element and a second binary data memory storage element of a memory for an implantable device.

FIG. 9B shows an exemplary implementation of the retention D flip-flop circuit shown in FIG. 9A.

FIG. 9C shows an exemplary implementation of a first type of inverter shown in FIG. 9B.

FIG. 9D shows an exemplary implementation of a second type of inverter shown in FIG. 9B.

FIG. 9E shows an exemplary implementation of a third type of inverter shown in FIG. 9B.

FIG. 10 shows an exemplary method for storing data using the memory elements shown in FIG. 9A.

### DETAILED DESCRIPTION

An implantable device provided herein may be configured to be implanted in a subject to monitor one or more physiological signals. In some embodiments, said physiological signals includes a pH, an amount of an analyte, an electrophysiological pulse, a temperature, a pressure, a potential, or combinations thereof. The implantable device may be configured to receive energy from an energy source that is separate from (i.e., external to) the device. The device containing the energy source may be a non-implantable device. Energy received from the external power source may be used to power the device. The implantable device may comprise a power management unit comprising an energy storage configured to store a portion of the energy received from the external power source. When the external power source stops providing energy to the implantable device, the implantable device may begin to draw on the energy stored in the energy storage.

An implantable device provided herein may comprise a memory configured to store a small amount of data that is essential to the device's continued operation when energy is not being provided by the external power source. The memory may require very small amounts of power to store said essential data and may be configured to retain data while other components of the device are disconnected from the power management unit.

The memory may comprise a plurality of binary data memory storage elements. As used herein, “binary data memory storage element” may refer to any device, apparatus, or system that is configured to store information encoded as binary digits (“bits”). One or more bits in a binary data memory storage element may be physically implemented using one or more physical systems that can exist in either of two physically distinct states. In some embodiments, a physical system used to store a bit of data in a binary data memory storage element may be a flip-flop circuit, an electrical switch, a circuit configured to allow two distinct current levels, a circuit configured to allow two distinct voltage levels, or a system having two distinct directions of magnetization.

In some embodiments, the memory of the implantable device may comprise two separate binary data memory storage elements, each of which may be independently coupled to a power management unit of the implantable device. The first binary data memory storage element may comprise one or more flip-flop circuits and may be configured to program and store data received from the device's physiological signal sensors when the device has sufficiently high amounts of power available. The second binary data memory storage element may comprise one or more retention latch circuits and may be configured to receive and store an essential portion of the data stored in the first binary data memory storage element when one or more conditions have been met. In some embodiments, said conditions may be associated with a total amount of power remaining in the device's energy storage or with an amount or type of data that has been collected. Once the essential data portion has been transmitted to the second binary data memory storage element, the implantable device may be configured to disconnect one or more elements—including the first binary data memory storage element—from the energy storage. The second binary data memory storage element may remain coupled to the energy storage, thereby ensuring that the essential data portion is retained while the rest of the implantable device is in a low power “sleep” mode.

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

A “binary data memory storage element” may refer to any device, apparatus, or system that is configured to store information encoded as binary digits (“bits”). One or more bits in a binary data memory storage element may be physically implemented using one or more physical systems that can exist in either of two physically distinct states. In some embodiments, a physical system used to store a bit of data in a binary data memory storage element may be a flip-flop circuit, an electrical switch, a circuit configured to allow two distinct current levels, a circuit configured to allow two distinct voltage levels, or a system having two distinct directions of magnetization.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

It is to be understood that one, some or all of the properties of the various embodiments described herein may be combined to form other embodiments of the present invention. The section headings used herein are for organizational purposes only and are not to be construed as limiting the subject matter described.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein. Further, sectional headings are provide for organizational purposes and are not to be considered limiting. Finally, the entire disclosure of the patents and publications referred in this application are hereby incorporated herein by reference for all purposes. To the extent that any reference incorporated by reference conflicts with the instant disclosure, the instant disclosure shall control.

### Implantable Device

An implantable device for sensing a physiological signal (e.g., pH, analyte concentration, pressure, strain, or temperature) in a subject (e.g., a human or an animal) may be powered through harvesting or transferring of energy from an energy source that is external to the device. The implantable device may be capable of receiving energy from the external energy source only when the external energy source is within a certain range of the implantable device. Furthermore, the implantable device may only be capable of storing a small amount of the energy that is transferred from the external energy source; as such, if the external energy source is moved out-of-range of the implantable device, the implantable device may have very low available power stored.

In some implementations, an implantable device may need to perform one or more operations (e.g., a measurement of a physiological signal) and retain data associated with said operations after the external energy source has been removed. The implantable device may need to operate and retain the data without depleting the energy storage on the device. The implantable devices described herein comprise a memory configured to store essential amounts of data while consuming very little energy from the device's energy storage.

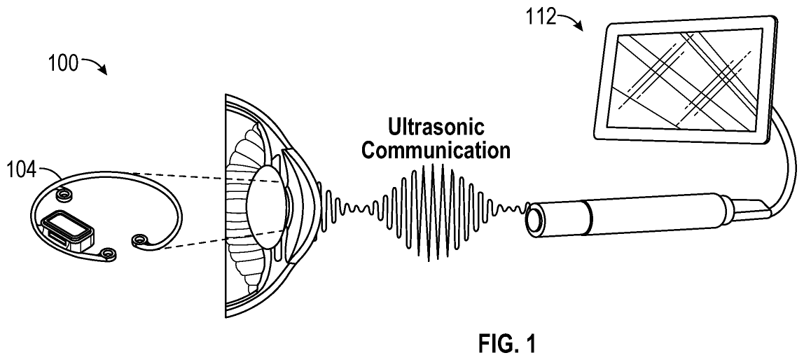

An exemplary implementation in which an implantable device may need to perform a measurement of a physiological signal and retain data associated with said measurement after the external energy source has been removed is an ocular implantable device for measuring an intraocular pressure. FIG. 1 shows a schematic of an exemplary system for measuring intraocular pressure. Specifically, FIG. 1 shows a system 100 comprising an implantable device 104 and an external device 112. Implantable device 104 may be configured to be implanted in a patient's eye and may comprise a pressure sensor configured to measure an intraocular pressure (IOP) of the eye. External device 112 may be configured to provide energy to and wirelessly communicate with implantable device 104.

In some embodiments, in order to provide energy to implantable device 104, external device 112 may need to be brought into contact with a location proximal to the patient's eye. When external device 112 is brought into contact with the location proximal to the patient's eye, external device 112 may apply a pressure to the patient's eye. This added pressure from the presence of external device 112 may alter the IOP of the eye. Thus, in order to ensure IOP measurements are not impacted by the presence of external device 112, implantable device 104 may be configured to measure IOP only after external device 112 has been removed from contact with the location proximal to the patient's eye.

When external device 112 is removed from the location proximal to the patient's eye, external device 112 may cease to provide implantable device 104 with energy. As such, implantable device 104 may comprise a small energy storage (e.g., a small capacitor) configured to store energy transmitted from external device 112 so that implantable device 104 may continue to function for a short length of time following the removal of external device 112. The energy storage on implantable device 104 may provide implantable device 104 with enough power to measure the IOP of the patient's eye and to store information associated with the IOP measurement in a memory element of implantable device 104.

In order for the IOP information to be analyzed by a user, the IOP information may need to be transferred off of implantable device 104. Implantable device 104 may be configured to wirelessly transmit the IOP information to external device 112. However, external device 112 may need to be repositioned in the location proximal to the patient's eye in order for the wireless transfer of IOP information to occur. Conventional memory elements may quickly deplete the energy storage of implantable device 104; as such, if the IOP information is not transferred to external device 112 within a short window of time, the energy stored on implantable device 104 may be depleted and the IOP information may be lost. Thus, in order to prevent the IOP information from being lost, implantable device 104 may require a memory element that is capable of retaining the IOP information for an extended period of time while consuming very low amounts of energy from implantable device 104′s energy storage.

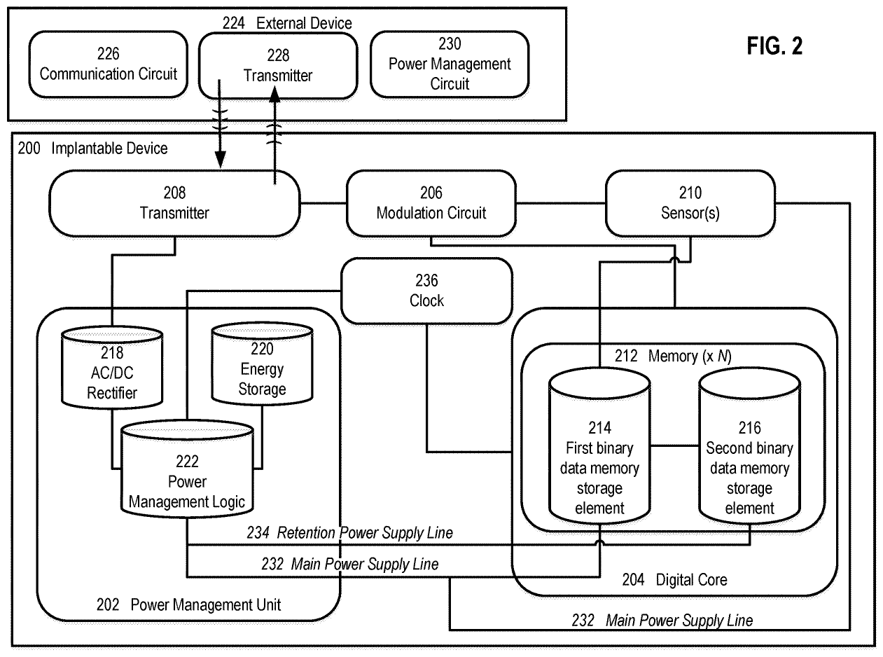

FIG. 2 shows an exemplary implantable device comprising an ultralow power memory, according to some embodiments of the present disclosure. Specifically, FIG. 2 illustrates an implantable device 200 comprising a power management unit 202 configured to store and distribute energy to components of implantable device 200, a digital core (also referred to throughout as a “digital circuit”) 204 configured to control one or more components of implantable device 200, a modulation circuit 206 configured to encode information in an electric current, a transmitter 208 configured to wirelessly transfer and receive energy from an external device 224, one or more sensors 210 configured to detect one or more physiological signals, and a memory 212 configured to store data received from the one or more sensors 210.

Implantable device 200 may be configured to be fully implantable in a subject (e.g., in a body part of a human or an animal). This may allow for careful monitoring of certain physiological signals in the subject. In some embodiments, implantable device 200 may be configured to be implanted in or attached to a tissue. For example, implantable device 200 may be configured to be implanted in or on a central nervous tissue, such as in the brain. In some embodiments, implantable device 200 may be configured to be implanted in or attached to an organ. For example, implantable device 200 may be configured to be implanted in an eye. In some embodiments, implantable device 200 may be configured to be implanted on a peripheral nerve (e.g., a splenic nerve) or neurovascular bundle.

In some embodiments, a total size of implantable device 200 may be small in order to allow implantable device to be more easily implanted in a subject. In some embodiments, implantable device 200 may be greater than or equal to 0.5, 1, 1.5, 2, 2.5, 3, 3.5, 4, 4.5 or 5 mm in its longest dimension. In some embodiments, implantable device 100 may be less than or equal to 0.5, 1, 1.5, 2, 2.5, 3, 3.5, 4, 4.5 or 5 mm in its longest dimension. In some embodiments, implantable device 200 may be between about 0.5-1.5, about 1.5-2.5, about 2.5-3.5, about 3.5-4.5, or about 4.5-5 mm in its longest dimension. In some embodiments, implantable device 200 may be greater than or equal to 0.5, 1, 1.5, 2, 2.5, 3, 3.5, 4, 4.5 or 5 mm 3 in total volume. In some embodiments, implantable device 200 may be less than or equal to 0.5, 1, 1.5, 2, 2.5, 3, 3.5, 4, 4.5 or 5 mm 3 in total volume. In some embodiments, implantable device 200 may be between about 0.5-1.5, about 1.5-2.5, about 2.5-3.5, about 3.5-4.5, or about 4.5-5 mm³ in total volume.

As shown in FIG. 2, implantable device 200 may be configured to couple to an external device 224. “External,” as used herein, refers to any region outside of implantable device 200 (i.e., separate from implantable device 200). In some embodiments, external device 224 may be external to the subject in which implantable device 100 is implanted. In some embodiments, external device 224 may be implanted in the subject along with implantable device 200, but may be separate from (i.e., external to) implantable device 200. External device 224 may be configured to wirelessly transmit signals to and receive signals from one or more components of implantable device 200.

In some embodiments, external device 224 may comprise a communication circuit 226, a transmitter 228, and a power management circuit 230. Transmitter 228 may be configured to wirelessly transmit energy to implantable device 200. In some embodiments, transmitter 228. Energy that is transmitted to implantable device 200 by transmitter 228 may include any form of energy that originates from outside of implantable device 200. In some embodiments, transmitter 228 may be configured to transmit energy to implantable device 200 in the form of ultrasonic waves or electromagnetic waves such as radio frequency waves. In some embodiments, transmitter 228 may be configured to transmit energy to implantable device 200 via induction or via a capacitive link.

Transmitter 208 on implantable device 200 may be configured to receive energy transmitted to implantable device 200 by transmitter 228 on external device 224. Transmitter 208 may be configured to convert the energy received from transmitter 228 into an electrical current. The electrical current generated by transmitter 208 may then be transmitted to power management unit 202.

In some embodiments, transmitter 228 may transmit energy to implantable device 200 in the form of ultrasonic waves. Transmitter 208 may comprise one or more ultrasonic transducers configured to convert ultrasonic waves received from transmitter 228 into electrical current. The one or more ultrasonic transducers may comprise a bulk piezoelectric transducer, a piezoelectric micro-machined ultrasonic transducer (PMUT), or a capacitive micro-machined ultrasonic transducer (CMUT).

In some embodiments, transmitter 228 may transmit energy to implantable device 200 in the form of electromagnetic waves. Transmitter 208 may comprise one or more antennas or one or more coils configured to convert electromagnetic waves (e.g., radio frequency waves) received from transmitter 228 into electrical current.

In some embodiments, transmitter 228 may transmit energy to implantable device 200 in the form of vibrational energy. Transmitter 208 may comprise one or more vibrational transducers configured to convert vibrations received from transmitter 228 into electrical current. In some embodiments, transmitter 208 may comprise one or more piezoelectric crystals configured to convert vibrations received from transmitter 228 into electrical current.

In some embodiments, transmitter 228 may transmit energy to implantable device 200 via induction. Transmitter 208 may comprise one a coil of wire. Transmitter 228 may also comprise a coil of wire. Power management circuit 230 may be configured to create an alternating current through its coil of wire. The alternating current in the external wire coil may generate a magnetic field. The generated magnetic field may induce an alternating current in the coil of transmitter 280.

In some embodiments, transmitter 228 may transmit energy to implantable device 200 via a capacitive link. Transmitter 208 and transmitter 228 may each comprise an electrode such that, when external device 224 is brought within a certain distance of implantable device 200, the electrode in transmitter 208 and the electrode in transmitter 228 form a capacitor. An oscillating voltage may be applied to the external electrode by power management circuit 230. The oscillating voltage may induce an oscillating electric field between the transmitter 228 electrode and the transmitter 208 electrode. This oscillating electric field may induce an oscillating voltage on the transmitter 208 electrode, thereby generating an alternating current that can be transmitted to power management unit 202.

Power management unit 202 may comprise an AC/DC rectifier 218, an energy storage 220, and a power management logic circuit 222. AC/DC rectifier 218 and energy storage 220 may be controlled by power management logic 222. Specifically, power management logic 222 may monitor signals received by AC/DC rectifier 218 to ensure implantable device 200 is operating at maximum efficiency. In addition, power management logic 222 may be configured to boost, buck, and/or regulate the voltage of AC/DC rectifier 218 in order to safely charge energy storage 220. Power management logic 222 may monitor the current, voltage, and/or energy of energy storage 220. In some embodiments, power management logic 222 may be configured to transmit signals comprising information about energy levels in energy storage 220 to digital core 204.

As described above, the electrical current generated by transmitter 208 may be an AC current. This generated AC current may be transmitted from transmitter 208 to AC/DC rectifier 218, which may convert it to a DC current. This DC current may then be transmitted to energy storage 220. Energy storage 220 may comprise one or more capacitors configured to store the energy carried by the current transmitted by transmitter 208.

As mentioned above, implantable device 200 may comprise one or more sensors 210 configured to sense one or more physiological signals. In some embodiments, the one or more sensors 210 may comprise a pressure sensor configured to measure a pressure within the subject (e.g., an intraocular pressure within the subject's eye). In some embodiments, the one or more sensors 210 may comprise one or more electrodes configured to detect an electrophysiological pulse (e.g., an electrophysiological pulse transmitted by a nerve, such as a peripheral nerve). In some embodiments, the one or more sensors 210 may comprise one or more sensors be configured to detect a concentration of an analyte (e.g., glucose or oxygen). In some embodiments, the one or more sensors 210 may comprise a temperature sensor configured to measure a temperature. In some embodiments, the one or more sensors 210 may comprise one or more sensors configured to measure a pH. In some embodiments, the one or more sensors 210 may comprise one or more sensors configured to measure a strain. In some embodiments, the one or more sensors 210 may comprise one or more sensors configured to measure an evoked action potential in a brain. In some embodiments, the one or more sensors 210 may comprise one or more sensors configured for measure a local field potential in a brain.

In some embodiments, a measurement of a physiological signal may be impacted if the measurement is conducted while external device 224 is providing energy to implantable device 200. Transmitter 228 in external device 224 may need to be brought within a close proximity of transmitter 208 in implantable device 200 in order to transfer energy to transmitter 208. In some embodiments, this may require external device 224 to be brought into direct contact with the patient. The close proximity or contact of external device 224 and/or the energy being transmitted by external device 224 may affect physiological conditions that the one or more sensors 210 are configured to measure.

In order to prevent errors in physiological signal measurements, the one or more sensors 210 may be configured to measure one or more physiological signals when external device 224 is not providing energy to implantable device 200. When a user wishes to conduct a physiological signal measurement, external device 224 may transfer energy to implantable device 200 until energy storage 220 has reached full capacity. External device 224 may then be caused to stop transferring energy to implantable device 200 (e.g., by a user removing external device 224 from a location proximal to the body part in which implantable device 200 is implanted).

In some embodiments, digital core 204 on implantable device 200 may be configured to monitor the amount of energy contained in energy storage 220. When energy storage 220 has reached full capacity, digital core 204 may cause modulation circuit 206 to encode, in an electrical current, instructions configured to cause external device 224 to stop transferring energy to implantable device 200. The information-encoded electrical current may be transmitted from modulation circuit 206 to transmitter 208. Transmitter 208 may convert the information-encoded electrical current to an energy signal (e.g., an ultrasonic wave or a radio wave). This energy signal may be transmitted to transmitter 228 of external device 224, which may convert the energy signal back to an information-encoded electrical current. This information-encoded current may be transmitted to communication circuit 226, which may extract the instructions encoded in the current and send a signal to the user so that the user may cause external device 224 to stop the transfer of energy to implantable device 200.

After energy storage 220 has been charged (e.g., reached full capacity or above some capacity threshold) and external device 224 has stopped transferring energy to implantable device 200, digital core 204 may be configured to cause the one or more sensors 210 to measure one or more physiological signals. Since energy is no longer being transferred from external device 224 to implantable device 200, the one or more sensors may draw power from energy storage 220 while measuring the one or more physiological signals. In some embodiments, the one or more sensors 210 may be electrically coupled to energy storage unit 220 through a main power supply line 232. Power management logic 222 may be configured to control the flow of energy through main power supply line 232 to the one or more sensors 210.

In some embodiments, after a physiological signal is measured by a sensor of the one or more sensors 210, digital core 204 may cause the sensor to write the physiological signal data into memory 212 for storage. In some embodiments, after a physiological signal is measured by a sensor of the one or more sensors 210, digital core 204 may cause the sensor to write the physiological signal data into memory 212 for storage. Memory 212 may be configured to store the physiological signal data until the data can be retrieved from implantable device 200 by external device 224.

In some embodiments, memory 212 may comprise a first binary data memory storage element 214 configured to store one or more bits of data and a second binary data memory storage element 216 configured to store one or more bits of data. First binary data storage element 214 may be electrically coupled to the one or more sensors 210 and to second binary data memory storage element 216. In some embodiments, digital core 204 may include N copies of memory 212, where N is an integer greater than or equal to one.

First binary data memory storage element 214 and second binary data memory storage element 216 may be electrically coupled to energy storage 220 of power management unit 202. In some embodiments, first binary data memory storage element 214 may be electrically coupled to energy storage 220 through main power supply line 232—e.g., through the same power supply line that provides energy to the one or more sensors 210. Second binary data memory storage element 216 may be electrically coupled to energy storage 220 through a separate retention power supply line 234.

In some embodiments, implantable device 200 may comprise a clock 236 configured to generate an oscillating signal. Clock 236 may be an oscillator (e.g., a crystal oscillator). Power management logic 222 may be configured to control the signal generated by clock 236. Digital core 204 may be electrically coupled to clock 236 and may be configured to transmit signals received from clock 236 to first binary data memory storage element 214 or second binary data memory storage element 216 to control where and when data is stored in memory 212.

When a sensor of the one or more sensors 210 measures a physiological signal, digital core 204 may cause the sensor to write data associated with the measured physiological signal into first binary data memory storage element 214. The one or more sensors 210 and first binary data memory storage element 214 may use energy from energy storage 210 as they are operated. Measurement of physiological signals and storage of physiological signal data in first binary data memory storage element 214 may proceed until digital core 204 detects that the amount of energy remaining in energy storage 220 has dropped below a predetermined threshold level. When digital core 204 detects that the energy remaining in energy storage 220 has dropped below the predetermined threshold level, digital core 204 may be configured to cause an essential portion of the data stored in first binary data memory storage element 214 to be transferred to second binary data memory storage element 216.

Just as external device 224 may need to be positioned proximal to the body part in which implantable device 200 is implanted in order to transfer energy to implantable device 200, external device 224 may need to be positioned proximal to the body part in which implantable device 200 in order to retrieve the physiological signal data that is stored on implantable device 200. If digital core 204 detects that the amount of energy remaining in energy storage 220 has dropped below the predetermined threshold level and that external device 224 has not yet been repositioned at the appropriate location, digital core 204 may be configured to cause implantable device 200 to enter a low power “sleep” mode. As described below, this low power “sleep” mode may allow an essential portion of the information stored in memory 212 to be retained until it can be retrieved by external device 224 without depleting the energy remaining in energy storage 220.

In some embodiments, the essential portion of the data may be data collected by the one or more sensors 210. In some embodiments, the essential portion of the data may comprise state information. In some embodiments, state information may comprise one or more flags or indicators that indicate that sensor data has been captured or that sensor data is valid.

The essential data transferred to second binary data memory storage element 216 may be data associated with a physiological signal measurement that was conducted at a certain point in time (e.g., the most recent measurement). Power management logic 222 may then be configured to disconnect main power supply line 232 from energy storage 220. Once main power supply line 232 has been disconnected, the one or more sensors 210 and first binary data memory storage element 214 will no longer draw energy from energy storage 220. Second binary data memory storage element 216, which is coupled to energy storage 220 through retention power supply line 234, may be the only component of implantable device 200 that continues to draw power from energy storage 220. In some embodiments, second binary data memory storage element 216 may be configured to retain the essential data while consuming a very small amount power from energy storage 220. At this point, implantable device 200 has entered the low power “sleep” mode.

At some point after implantable device 200 has entered the low power “sleep” mode, a user may decide to retrieve the data stored on implantable device 200. External device 224 may be repositioned in the location proximal to the patient's body part. In order to ensure implantable device 200 has enough energy to transmit the stored data to external device 224, external device 224 may be configured to transmit energy to implantable device 200 using transmitter 228. Transmitter 208 may receive the energy transmitted by transmitter 228 and convert the energy to an electric current. This current may be transmitted to energy storage 220, thereby causing the amount of energy contained in energy storage 220 to increase. Once digital core 204 detects that the amount of energy contained in energy storage 220 has surpassed the predetermined threshold level, power management circuit 222 may be configured cause implantable device 200 to “wake up” by reconnecting main power supply line 232 to energy storage 220.

After implantable device 200 has “reawakened,” digital core 204 may be configured to cause a signal to be transmitted to external device 224 that instructs external device 224 to stop transmitting energy to implantable device 200. While external device 224 remains in the position proximal to the patient's body part but is not transmitting energy to implantable device 200, digital core 204 may be configured to cause modulation circuit 206 to modulate an electric current received from power management unit 202 in order to encode the essential data stored in second binary data memory storage element 216 in the current. In some embodiments, digital core 204 may be configured to cause modulation circuit 206 to encode the essential data stored in second binary data memory storage element 216 only if the essential data comprises state information indicating that the sensor data collected by sensors 210 before device 200 entered the low power “sleep” mode is valid. Modulation circuit 206 may transmit the data-encoded current to transmitter 208. Transmitter 208 may convert the data-encoded current into an energy signal (e.g., ultrasonic waves or radio waves) and transmit the energy signal to external device 224. Transmitter 228 of external device 224 may receive the energy signal and convert it back to a data-encoded electric current. This data-encoded current may be transmitted to communication circuit 226 for further processing.

### Ultralow Power Memory

Implantable devices for sensing physiological signals (e.g., implantable device 100 shown in FIG. 1) may comprise a power management unit including an energy receiver configured to receive energy from a power source that is external to the device. The power management unit may comprise an energy storage (e.g., one or more capacitors) capable of storing small amounts of power. If the external power source is not providing energy to the power management unit, the implantable device may have very low levels of available power upon which it can draw. In some embodiments, for example, the total size of an implantable device may be restricted in order to allow the device to be implanted in a small region of the body (e.g., in an eye). The capacity of the implantable device's energy storage may be proportional to the size of the device. The capacitance of a capacitor, for instance, is directly proportional to the size of the capacitor. Limiting the size of the implantable device restricts the size of the device's energy storage and, as a result, limits the device's energy storage capacity.

After the power management unit stops receiving energy from the external power source, the implantable device may need to retain data for extended periods of time without depleting the energy stored in the power supply. As such, the implantable device may comprise a memory element (e.g., memory 212 shown in FIG. 2) configured to retain small amounts of data using very little energy while other portions of the device are in the low power “sleep” mode.

In some embodiments, the memory element may comprise a plurality of binary data memory storage elements. A binary data memory storage element may be any device, apparatus, or system that is configured to store information encoded as binary digits (“bits”). One or more bits in a binary data memory storage element may be physically implemented using one or more physical systems that can exist in either of two physically distinct states. In some embodiments, a physical system used to store a bit of data in a binary data memory storage element may be a flip-flop circuit, an electrical switch, a circuit configured to allow two distinct current levels, a circuit configured to allow two distinct voltage levels, or a system having two distinct directions of magnetization.

In some embodiments, one or more of the binary data memory storage elements in the memory element may be or may comprise a flip-flop circuit and/or a latch circuit. For example, in some embodiments, a memory element for an implantable device may comprise a retention cell or a retention flip-flop circuit. A retention cell in a memory element for an implantable device may be any retention cell that is readily available in standard library cells. In such embodiments, a first binary data memory storage element may comprise a conventional flip-flop circuit while a second binary data memory storage element may comprise a retention latch circuit.

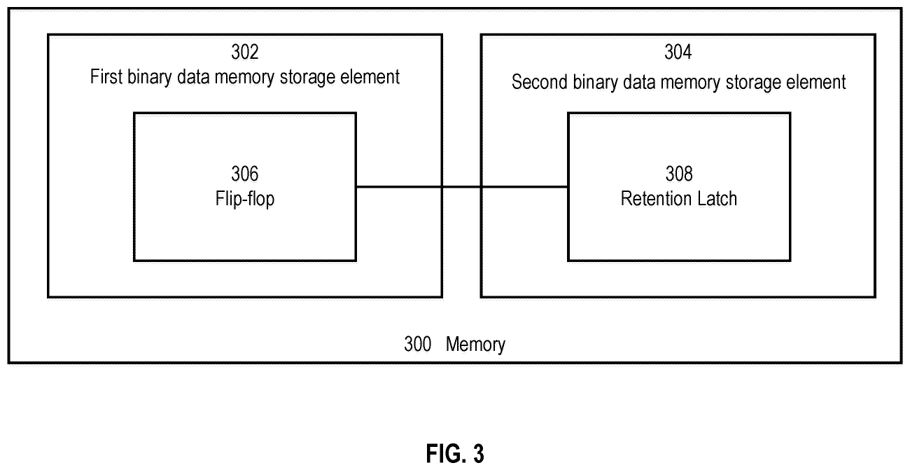

FIG. 3 shows an exemplary memory element for an implantable device according to some embodiments of the present disclosure. Specifically, FIG. 3 illustrates a memory 300 comprising a first binary data memory storage element 302 and a second binary data memory storage element 304. In some embodiments, memory 300 may be a memory for an implantable device for sensing one or more physiological signals such as implantable device 200 shown in FIG. 2.

Memory 300 may be electrically coupled to and configured to receive energy from an energy storage on the implantable device. The energy storage may include one or more features of energy storage 220 of implantable device 200 shown in FIG. 2. In some embodiments, first binary data memory storage element 302 and second binary data memory storage element 304 may be independently electrically coupled to the energy storage through separate power supply lines.

First binary data memory storage element 302 may be electrically coupled to the energy storage through a main power supply line (e.g., main power supply line 232). Second binary data memory storage element 304 may be electrically coupled to the energy storage through a separate retention power supply line (e.g., retention power supply line 234). The main power supply line may be configured to transmit a first amount of power from the energy storage to first binary data memory storage element 302. The retention power supply line may be configured to transmit a second amount of power from the energy storage to second binary data memory storage element 304. In some embodiments, the first amount of power transmitted by the main power supply line may be greater than or equal to the second amount of power transmitted by the retention power supply line. In some embodiments, the second amount of power may be less than or equal to 10, 50, 100, 500, 1000, or 104 picowatts.

In some embodiments, the implantable device may comprise a digital core (i.e., a digital circuit) that is configured to control power output from the energy storage. In particular, the digital core may be configured to disconnect the main power supply line from the energy storage whenever the digital core detects that one or more conditions have been met. In some embodiments, the one or more conditions may be associated with an amount of energy contained in the energy storage at a given point in time. When the digital core disconnects the main power supply line from the energy storage, the implantable device may enter a low power “sleep” mode wherein first binary data memory storage element 302 (along with any other elements of the implantable device that are coupled to the energy storage through the main power supply line) draws no power from the energy storage. The retention power supply line, on the other hand, may at all times remain connected to power management unit 210 through retention power supply line 214, thereby ensuring that second binary data memory storage element 304 receives a constant supply of energy.

Memory 300 may be configured to store data received from one or more sensors of the implantable device. The one or more sensors may be configured to sense one or more physiological signals (see, e.g., the one or more sensors 210 shown in FIG. 2).

In some embodiments, first binary data memory storage element 302 may be configured to receive and store data transmitted from the one or more sensors. In some embodiments, the one or more sensors may be electrically coupled to the energy storage of the implantable device through the main power supply line. Thus, the sensors may only transmit data to first binary data memory storage element 302 for storage when the main power supply line is connected to the energy storage, i.e., when the implantable device is “awake.”

First binary data memory storage element 302 may be or may comprise one or more flip-flop circuits 306. Each flip-flop circuit 306 may have two stable states. The two stable states of each flip-flop circuit 306 may be configured to store binary data. In some embodiments, first binary data memory storage element 302 may be configured to store one or more bits of the data received from the one or more sensors by changing the states of the one or more flip-flop circuits 306. In some embodiments, first binary data memory storage element 302 may be configured to store an amount of data greater than or equal to the amount of data that second binary data memory storage element 304 can store.

In some embodiments, a flip-flop circuit 306 may comprise one or more SR (“set-reset”) flip-flops, one or more D (“data” or “delay”) flip-flops, one or more T (“toggle”) flip-flops, and one or more JK flip-flops. A flip-flop circuit 306 may be implemented using one or more pairs of cross-coupled inverting elements. In some embodiments, a flip-flop circuit 306 may be implemented using one or more transistors, one or more inverters, and/or one or more logic gates.

In some embodiments, when the digital core (i.e., a digital circuit) of the implantable device has detected the one or more conditions indicating that the implantable device should be caused to enter the low power “sleep” mode, the digital core may be configured to cause memory 300 to transfer an essential portion of the data stored in first binary data memory storage element 302 to second binary data memory storage element 304. In some embodiments, the essential portion of the data that is transferred from first binary data memory storage element 302 to second binary data memory storage element 304 may comprise data associated with a measurement that was performed by a sensor at a specific moment in time, such as data associated with the most recently performed measurement.

Second binary data memory storage element 304 may comprise one or more retention latch circuits 308. Each retention latch circuit 308 may have two stable states. The two stable states of each retention latch circuit 308 may be configured to store binary data. In some embodiments, second binary data memory storage element 304 may be configured to store one or more bits of the essential data portion received from first binary data memory storage element 302 by changing the states of the one or more retention latch circuits 308. Second binary data memory storage element 304 may include fewer retention latch circuits 308 than the number of flip-flop circuits 306 included in first binary data memory storage element 302. Thus, second binary data memory storage element 304 may have a smaller storage capacity than first binary data memory storage element 302. In some embodiments, second binary data memory storage element 304 may be capable of storing between about 1-10, about 10-50, about 10-100, or about 10-500 bits.

Like flip-flop circuits 306, in some embodiments, a retention latch circuit 308 may comprise one or more SR (“set-reset”') flip-flops, one or more D (“data” or “delay”) flip-flops, one or more T (“toggle”) flip-flops, and one or more JK flip-flops. A retention latch circuit 308 may be implemented using one or more pairs of cross-coupled inverting elements. In some embodiments, a retention latch circuit 308 may be implemented using one or more transistors, one or more inverters, and/or one or more logic gates. In some embodiments, second binary data memory storage element 304 may be configured to retain the essential portion of the data until the digital core (i.e., a digital circuit) of the implantable device detects that the implantable device has begun receiving energy from an external power source once again. At this point, the digital core may reconnect the main power supply line to the energy storage, thereby causing the implantable device to enter a full-power “awake” mode. After the implantable device has re-entered the “awake” mode, the digital core may cause the essential data portion that is stored in retention latch circuits 308 of second binary data memory storage element 304 to be transmitted to an external device. A modulation circuit (e.g., modulation circuit 206 shown in FIG. 2) may be configured to encode the essential data portion in an electrical current flowing through a transmitter (e.g., transmitter 208 shown in FIG. 2) of the implantable device. In some embodiments, the transmitter may then emit backscatter waves (e.g., ultrasonic backscatter waves or radio frequency backscatter waves) that include the encoded data. The emitted backscatter waves may be received by a transmitter of an external device (e.g., transmitter 228 of external device 224 shown in FIG. 2). After the backscatter waves are received by the external device, the essential data may be extracted from the backscatter waves.

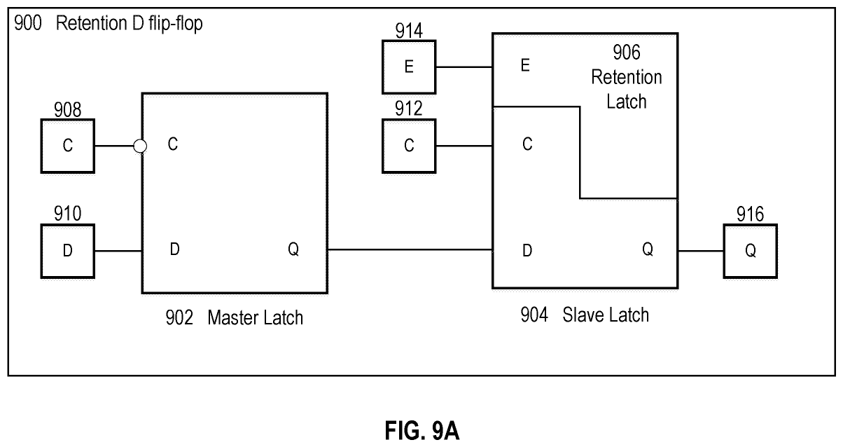

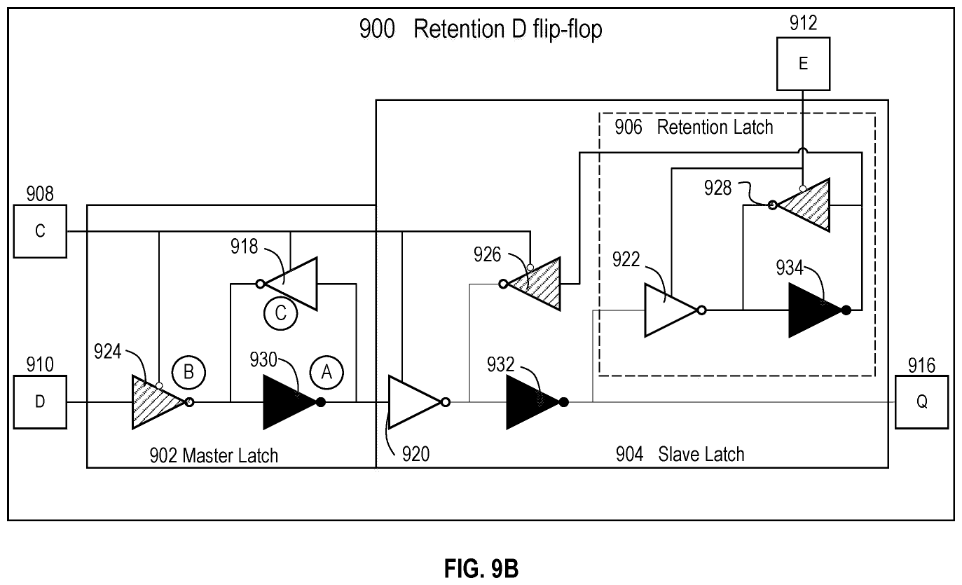

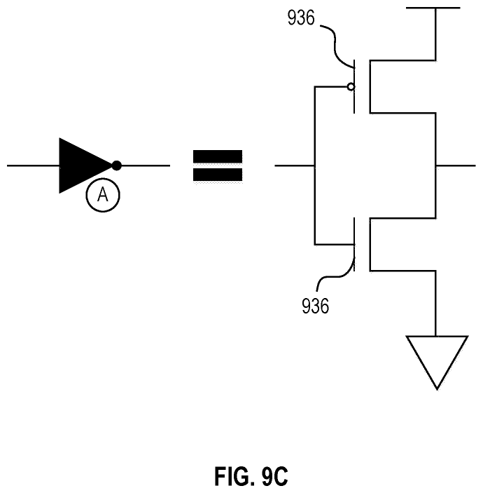

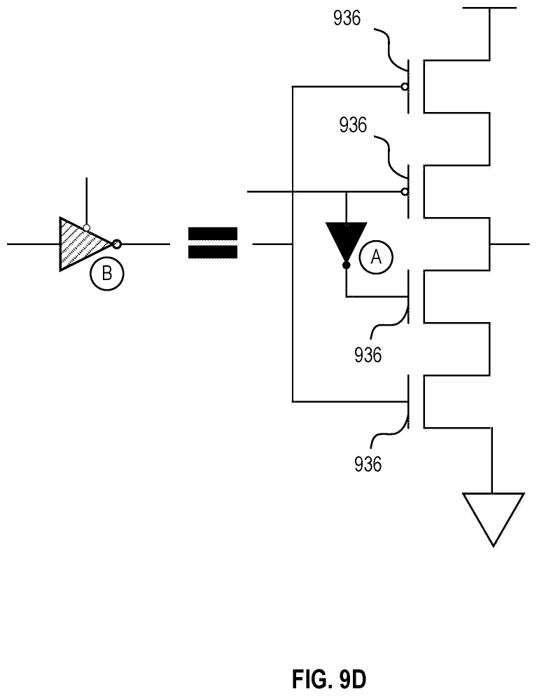

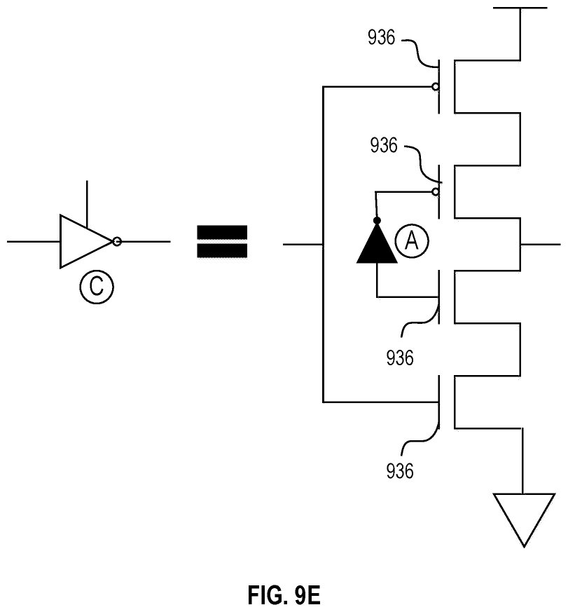

An exemplary implementation of a first binary data memory storage element and a second binary data memory storage element that may form components of a memory (e.g., memory 300) of an implantable device is shown in FIG. 9A. FIG. 9A shows a retention D flip-flop 900 comprising a master latch circuit 902 connected in series to a slave latch circuit 904 that contains a retention latch circuit 906.

In some embodiments, master latch 902 and slave latch 904 may be coupled to a power management unit of the implantable device (e.g., power management unit 202 of implantable device 200 shown in FIG. 2) through a main power supply line. Retention latch 906 may be additionally coupled to the power management unit through a retention power supply line that is separate from the main power supply line.

Master latch 902 may be configured to receive a first input 908 and a second input 910. The second input 910 to master latch 902 may comprise data (D) received from one or more sensors of the implantable device. The first input 908 to master latch 902 may be a “Clock” (C) signal that is configured to control the loading of data from the one or more sensors to master latch 902 through the second input 910. In some embodiments, the implantable device may include a clock source (e.g., clock 236 of implantable device 200 shown in FIG. 2) configured to generate the Clock signal. The Clock signal generated by the clock source may be controlled by the power management logic of the implantable device (e.g., power management logic 222 shown in FIG. 2). The digital core (e.g., digital core 204 shown in FIG. 2) may control when the Clock signal is received by flip-flop 900.

Like master latch 902, slave latch 904 may be configured to receive a Clock signal 912. Clock signal 912 received by slave latch 904 may control the loading of data from master latch 902 to slave latch 904. Retention latch 906 may be configured to receive an input 914. The input 914 to retention latch 906 may be an “Enable” (E) signal that is configured to cause flip-flop 900 to control when data should be held in retention latch 906. Clock signal 912 and Enable signal 914 may be controlled by the digital core of the implantable device (e.g., digital core 204 shown in FIG. 2).

When the implantable device is operating in a full-power “awake” mode, the digital core of the implantable device may hold Enable signal 914 in a high (E=1) state. When Enable signal 914 is high, retention D flip-flop 900 may act as a standard D flip-flop. Specifically, when enable signal 914 is high, slave latch 904 (which contains retention latch 906) may act identically to a slave latch that does not contain a retention latch structure.

While holding Enable signal 914 in a high state, the digital core may control the loading and holding of data in master latch 902 and slave latch 904 by controlling the Clock signal 908 and Clock signal 912. In some embodiments, when the digital core holds the Clock in a low (C=0) state, master latch 902 may load data from one or more sensors of the implantable device through the second master latch input 910 and slave latch 904 may hold data. In some embodiments, when digital core raises the Clock to a high (C=1) state, master latch 902 may hold data while slave latch 904 loads data.

When the implantable device is operating in a low power “sleep” mode, the digital core of the implantable device may hold Enable signal 914 in a low (E=0) state. When Enable signal 914 is low, retention latch 906 may be configured to hold its value regardless of the value of the Clock signal that is input to slave latch 904. In some embodiments, the digital core may be configured to pull Enable signal 914 from a high state to a low state upon determining that one or more conditions associated with the energy available in the device's power storage have been met.

FIG. 9B shows an exemplary implementation of retention D flip-flop 900 shown in FIG. 9A. As shown, master latch 902 may comprise an input inverter 918, a feedback inverter 924, and an output inverter 930; slave latch 904 may comprise an input inverter 920, a feedback inverter 926, and an output inverter 932; and retention latch 906 may comprise an input inverter 922, a feedback inverter 928, and an output inverter 934.

The feedback inverters 924 and 926 in master latch 902 and slave latch 904 may be configured to be enabled by Clock signal 908. The feedback inverter 928 in retention latch 906 may be configured to be enabled by Enable signal 912. When Enable signal 912 is low (E=0), input inverter 922 of retention latch 906 may be disabled, while feedback inverter 928 may be enabled. This may isolate retention latch 906 from the other components of flip-flop 900 and allow retention latch 906 to hold its value regardless of the value of Clock signal 908. When Enable signal 912 is high (E=1), feedback inverter 928 of retention latch 906 may be disabled, while input inverter 922 of retention latch 906 may be enabled. This may allow flip-flop 900 to operate identically to a simple D flip-flop (i.e., a D flip-flop without a retention latch structure).

Exemplary implementations of the output inverters, feedback inverters, and output input are shown in FIGS. 9C-9E, respectively. As shown, in some embodiments, the inverters may comprise one or more transistors 936. In some embodiments, the inverters used to form retention latch 906 may be implemented using transistor devices with low leakage current in order to reduce the amount of power consumed by retention latch 906.

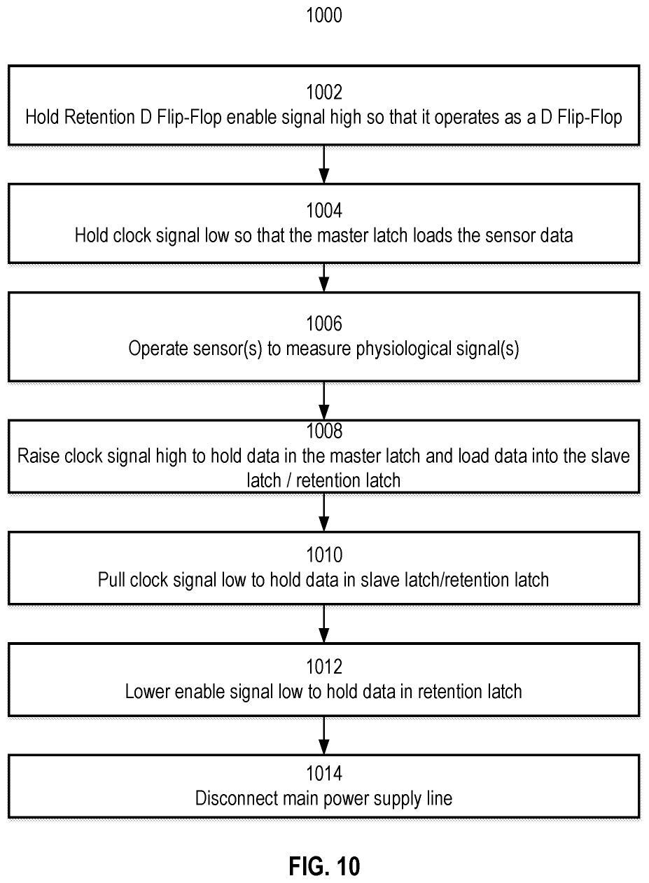

FIG. 10 shows an exemplary method 1000 for storing data using the retention D flip-flop 900 shown in FIG. 9A. In some embodiments, method 1000 may be performed after the implantable device has determined that it is no longer receiving power from an external power source (e.g., because the external power source has been removed from a vicinity of the implantable device).

In a step 1002, after the implantable device has been commanded to execute a measurement of one or more physiological signals, the digital core of the implantable device may raise an enable signal (e.g., enable signal 912) of the retention latch in a high (E=1) state so that the retention D flip-flop operates as a simple D flip-flop. Next, in a step 1004, while the enable signal is held high, the digital core may lower the clock signal to a low (C=0) state so that the master latch can load data from the one or more sensors. While the enable signal is held high and the clock signal is held low, method 1000 may move to step 1006, wherein the digital core may operate the device's sensors to measure one or more physiological signals.

After the sensors have measured the physiological signals in step 1006, method 1000 may proceed to step 1008, wherein the digital core may raise the clock signal to a high (C=1) state to cause data to be held in the master latch and loaded into the slave latch. Method 1000 may then move to a step 1010, wherein the digital core may lower the clock signal once again in order to cause data to be held in the slave latch. Once the data is held in the slave latch, method 1000 may proceed to step 1012, wherein the digital core may lower the enable signal to a low (E=0) state to cause data to be held in the retention latch. At this point, method 1000 may proceed to step 1014, wherein the main power supply line (i.e., the power supply line that supplies energy to the implantable device while the device is operating in a full-power “awake” mode) may be disconnected from the device's power storage, and the device may enter a low power “sleep” mode. While the device is in its low power “sleep” mode, the digital core may hold the enable signal in the low (E=0) state in order to ensure that the data continues to be held in the retention latch.

### The External Device

As described above, the implantable devices described herein receive energy from and wirelessly communicate with an external device. In some embodiments, the external device may be an interrogator comprising one or more transmitters (e.g., ultrasonic transducers or RF antennas) configured to transmit energy to and receive energy from the implantable device. The interrogator may comprise a power management circuit configured to provide power to the one or more transmitters and a communication circuit configured decode wireless communications received from the implantable device.

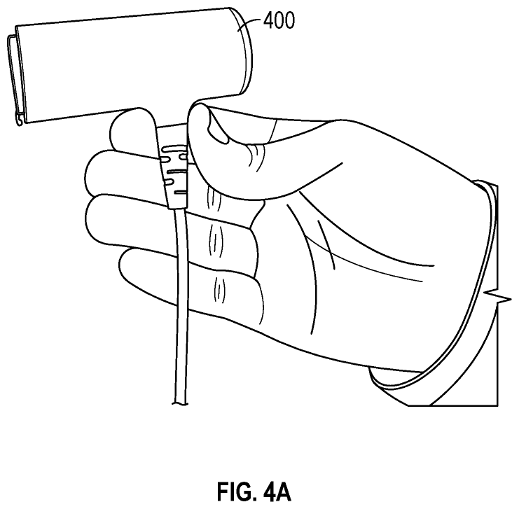

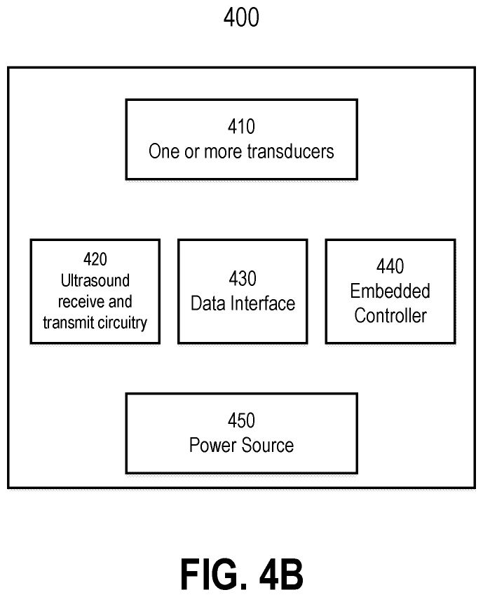

FIG. 4A shows an exemplary interrogator for providing energy to and communicating with an implantable device for measuring a physiological signal (for example, an ocular implantable device for measuring an intraocular pressure). An exemplary schematic of the exemplary interrogator (i.e., external device) for providing energy to and communicating with the implantable device is shown in FIG. 4B.

The interrogator of FIGS. 4A-4B may be configured to wirelessly communicate with an implantable device (e.g., the intraocular implantable device). The external device 400 may include one or more transducers 410 for wireless communication. In some embodiments, the one or more transducers 410 may include an ultrasonic transducer. The ultrasonic transducer may be configured to ultrasonically couple to skin (e.g., for an intraocular implantable device system, the skin of an eyelid, skin over a brow bone, skin over a nasal bone, or skin over an eye socket) to facilitate ultrasonic communication between the external device and the implantable device (e.g., a device mounted on or within an eye). In some embodiments, an ultrasound coupling gel or an alternative couplant may be used to ultrasonically couple the external device to the skin.

In some embodiments, the external device 400 may include ultrasound receive and transmit circuitry 420, a data interface 430, an embedded controller 440, and a power source 450. In some embodiments, the device may be configured to rely on power transmission from the external device. The power transmission from the external device may be used to power the device to initiate physiological signal measurements collected by the one or more sensors of the implantable device. In some embodiments, the one or more transducers 410 of the external device may be configured to transmit instructions to the implantable device. The instructions from the external device may instruct the device to reset itself, enter a specific mode, set device parameters, or begin a transmission sequence.

Physical contact between the patient and the external device (e.g., between the eye/eyelid of the patient and the external device) enables the external device to receive measurements from the implanted device. Optionally, the external device may be controlled using a separate computer system, such as a mobile device (e.g., a smartphone or a table). The computer system can wirelessly communicate to the interrogator, for example through a network connection, a radio frequency (RF) connection, or Bluetooth. The computer system may, for example, turn on or off the external device or analyze information encoded in backscatter waves (e.g., ultrasonic backscatter waves or radio frequency backscatter waves) received by the external device.

The implantable device and the external device may wirelessly communicate with each other, for example using ultrasonic waves or radio frequency waves. The communication may be a one-way communication (for example, the external device transmitting information to the implantable device, or the implantable device transmitting information to the external device), or a two-way communication (for example, the interrogator transmitting information to the device, or the device transmitting information to the interrogator). Information transmitted from the device to the external device may rely on, for example, a backscatter communication protocol. For example, the external device may transmit ultrasonic waves to the implantable device, which may emit backscatter waves that encode the information. The external device can receive the backscatter waves and decipher the information encoded in the received backscatter waves.

In some embodiments, the one or more ultrasonic transducers of the implantable device may include a piezoelectric crystal configured to receive commands from ultrasonic energy transmitted from the external interrogator. The implantable device may decode pulse interval encoded commands transmitted from the external interrogator and may passively transmit data to the external device via amplitude-modulated, backscatter communication. In some embodiments, the implanted device receives ultrasonic waves from the external device through one or more ultrasonic transducers on the implantable device, and the received waves can encode instructions for operating the implantable device. For example, vibrations of the ultrasonic transducer(s) on the device generate a voltage across the electric terminals of the transducer, and current flows through the device, including the integrated circuit. The current (which may be generated, for example, using one or more ultrasonic transducers) can be used to charge an energy storage circuit.

In some embodiments, ultrasonic backscatter is emitted from the implantable device, which can encode information relating to the device. In some embodiments, a device is configured to detect a physiological signal (e.g., intraocular pressure), and information regarding the detected physiological signal can be transmitted to the external device by the ultrasonic backscatter. To encode physiological signal information in the backscatter, current flowing through the ultrasonic transducer(s) of the device is modulated as a function of the encoded information, such as a measured physiological condition. In some embodiments, modulation of the current can be an analog signal, which may be, for example, directly modulated by the detected physiological condition. In some embodiments, modulation of the current encodes a digitized signal, which may be controlled by a digital core (i.e., a digital circuit) in the integrated circuit. The backscatter is received by an external device (which may be the same or different from the external device that transmitted the initial ultrasonic waves). The information can thus be encoded by changes in amplitude, frequency, or phase of the backscattered ultrasound waves.

In some embodiments, the ultrasound communication does not raise the temperature of any part of the eye more than about 1.5° C. at any time, in accordance with ISO 14708-01:2014 clause 17 which stipulates any surface of the implant shall not exceed a temperature increase of 2° C.

In some embodiments, the ultrasound communication may be established when the piezoelectric crystal of the device is about 5 mm+/−20 % distance from the external device. In some embodiments, the ultrasound communication may be established when a surface of the piezoelectric crystal is at most about a 3 mm, 5 mm, 7 mm, or 9 mm distance from a surface of the external device configured to be in physical contact with the patient (e.g., configured to touch skin of an eyelid, skin over a brow bone, skin over a nasal bone, or skin over an eye socket). In some embodiments, the ultrasound communication may be established when a surface of the piezoelectric crystal is at least about 1 mm, 2 mm, or 3 mm distance from the interrogator configured to be in physical contact with the patient (e.g., configured to touch skin of an eyelid, skin over a brow bone, skin over a nasal bone, or skin over an eye socket). In some embodiments, the ultrasound communication may be established when a surface of the piezoelectric crystal is about 1-9 mm, 2-7 mm, or 3-5 mm distance from the external device configured to be in physical contact with the patient (e.g., configured to touch skin of an eyelid, skin over a brow bone, skin over a nasal bone, or skin over an eye socket). Once established, the ultrasound communication may tolerate typical involuntary movement by the patient for the brief duration of the physiological signal measurement (for example, involuntary eye movement for the brief duration of the IOP measurement).

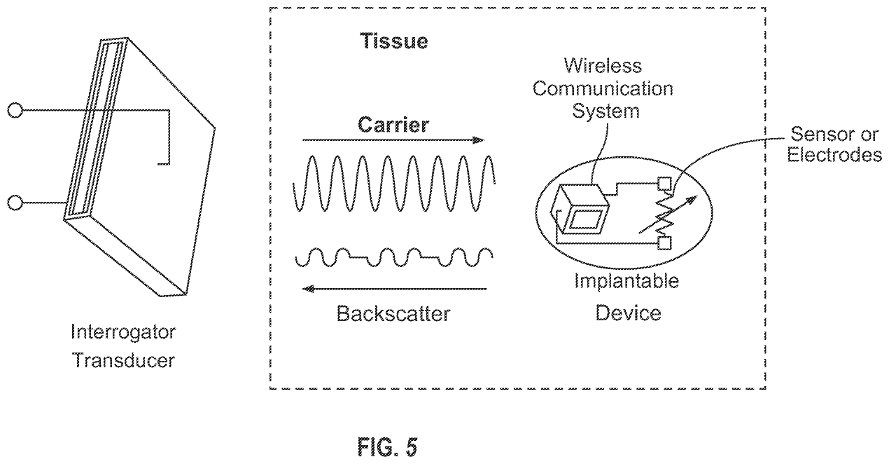

FIG. 5 shows an exemplary interrogator in communication with an exemplary implantable device for measuring a physiological signal (e.g., an ocular implant for measuring intraocular pressure). The external ultrasonic transceiver emits ultrasonic waves (“carrier waves”), which can pass through tissue. The carrier waves cause mechanical vibrations on the ultrasonic transducer (e.g., a bulk piezoelectric transducer, a PUMT, or a CMUT). A voltage across the ultrasonic transducer is generated, which imparts a current flowing through an integrated circuit on the implantable device. The current flowing through to the ultrasonic transducer causes the transducer on the implantable device to emit backscatter ultrasonic waves. In some embodiments, the integrated circuit modulates the current flowing through the ultrasonic transducer to encode information, and the resulting ultrasonic backscatter waves encode the information. The backscatter waves can be detected by the external device and can be analyzed to interpret information encoded in the ultrasonic backscatter.

The instructions from the external device to the device can be carried by the ultrasonic carrier. Specifically, the ultrasonic carrier generated by the ultrasonic transducer of the external device may include a series of ultrasonic pulses that have a varying number of carrier periods. The number of carrier periods encode information specific to the device. For example, based on the number of carrier periods, the information may include instructions for the device to begin a data transmission sequence. The transmission sequence can include steps for measuring physiological signal data (e.g., IOP data) and encoding the physiological signal data as ultrasonic backscatter. The encoding includes backscattering the physiological signal data on the ultrasonic carrier to modulate the electrical current and converting the modulated current to ultrasonic backscatter for transmission to the external device. The number of carrier periods may encode other information related to the device. For example, the information may include instructions for the device to reset itself, enter a specific mode, or set device parameters.

Communication between the external device and the implantable device can use a pulse-echo method of transmitting and receiving ultrasonic waves. In the pulse-echo method, the interrogator transmits a series of interrogation pulses at a predetermined frequency, and then receives backscatter echoes from the implanted device. In some embodiments, the pulses are square, rectangular, triangular, sawtooth, or sinusoidal. In some embodiments, the pulses output can be two-level (GND and POS), three-level (GND, NEG, POS), 5-level, or any other multiple level (for example, if using 24-bit DAC). In some embodiments, the pulses are continuously transmitted by the external device during operation. In some embodiments, when the pulses are continuously transmitted by the interrogator a portion of the transducers on the interrogator are configured to receive ultrasonic waves and a portion of the transducers on the interrogator are configured to transmit ultrasonic waves. Transducers configured to receive ultrasonic waves and transducers configured to transmit ultrasonic waves can be on the same transducer array or on different transducer arrays of the external device. In some embodiments, a transducer on the external device can be configured to alternatively transmit or receive the ultrasonic waves. For example, a transducer can cycle between transmitting one or more pulses and a pause period. The transducer is configured to transmit the ultrasonic waves when transmitting the one or more pulses and can then switch to a receiving mode during the pause period.

In some embodiments, the backscattered waves are digitized by the implantable device. For example, the implantable device can include an oscilloscope or analog-to-digital converter (ADC) and/or a memory, which can digitally encode information in current (or impedance) fluctuations. The digitized current fluctuations, which can encode information, are received by wireless communication system, which then transmits digitized ultrasonic waves. The digitized data can compress the analog data, for example by using singular value decomposition (SVD) and least squares-based compression. In some embodiments, the compression is performed by a correlator or pattern detection algorithm. The backscatter signal may go through a series of nonlinear transformation, such as 4th order Butterworth band pass filter rectification integration of backscatter regions to generate a reconstruction data point at a single time instance. Such transformations can be done either in hardware (i.e., hard-coded) or in software.

In some embodiments, the digitized signal compresses the size of the analog signal. The decreased size of the digitized signal can allow for more efficient reporting of information encoded in the backscatter. By compressing the size of the transmitted information through digitization, potentially overlapping signals can be accurately transmitted.

The wireless communication system, which can communicate with a separate device (such as an external interrogator or another device). For example, the wireless communication may be configured to receive instructions for emitting ultrasonic backscatter associated with measured physiological signal data (e.g., IOP data) from the one or more sensors. The wireless communication system can include, for example one or more ultrasonic transducers. The wireless communication system may also be configured to receive energy (for example, through ultrasonic waves) from another device, which can be used to power the implantable device.

In addition to providing the device with instructions, in some embodiments, the ultrasonic carrier from the interrogator may transmit vibrational energy configured to power the device. That is, the ultrasonic pulses of the ultrasonic carrier is delivered to the device at a frequency suitable for imparting energy to power the ASIC.

In some embodiments, the implantable device can also be operated to transmit information (i.e., uplink communication), which can be received by the interrogator, through the wireless communication system. In some embodiments, the wireless communication system is configured to actively generate a communication signal (e.g., ultrasonic waves) that encode the information. In some embodiments, the wireless communication system is configured to transmit information encoded on backscatter waves (e.g., ultrasonic backscatter waves). Backscatter communication provides a lower power method of transmitting information, which is particularly beneficial for small devices to minimize energy uses. By way of example, the wireless communication system may include one or more ultrasonic transducers configured to receive ultrasonic waves and emit an ultrasonic backscatter, which can encode information transmitted by the implantable device. Current flows through the ultrasonic transducer, which can be modulated to encode the information. The current may be modulated directly, for example by passing the current through a sensor that modulates the current, or indirectly, for example by modulating the current using a modulation circuit based on a detected physiological condition such as IOP.

The information wirelessly transmitted using the wireless communication system can be received by an interrogator. In some embodiments, the information is transmitted by being encoded in backscatter waves (e.g., ultrasonic backscatter). The backscatter can be received by the interrogator, for example, and deciphered to determine the encoded information. Additional details about backscatter communication are provided herein, and additional examples are provided in WO 2018/009905; WO 2018/009908; WO 2018/0091010; WO 2018/009911; WO 2018/009912; International Patent Application No. PCT/US2019/028381; International Patent Application No. PCT/US2019/028385; and International Patent Application No. PCT/2019/048647; each of which is incorporated herein by reference for all purposes. The information can be encoded by the integrated circuit using a modulation circuit. The modulation circuit is part of the wireless communication system and can be operated by or contained within the integrated circuit.

### Methods for Storing and Retrieving Data on an Implantable Device

As described in previous sections, an implantable device for sensing one or more physiological signals may comprise a memory element configured to retain small amounts of data using very little energy while other portions of the device are in the low power “sleep” mode. One or more processors (e.g., the digital core) of the implantable device may be configured to control how data detected by one or more sensors of the implantable device is stored based on a current power status of the device (for example, based on whether the implantable device is currently receiving power from an external power source or based on an amount of energy currently available in an energy storage of the implantable device). In some embodiments, one or more processors (e.g., the digital core) of the implantable device may be configured to selectively disconnect and reconnect one or more elements of the memory of the implantable device from a power management unit of the implantable device in order to conserve energy when the device is not receiving power from an external power source.

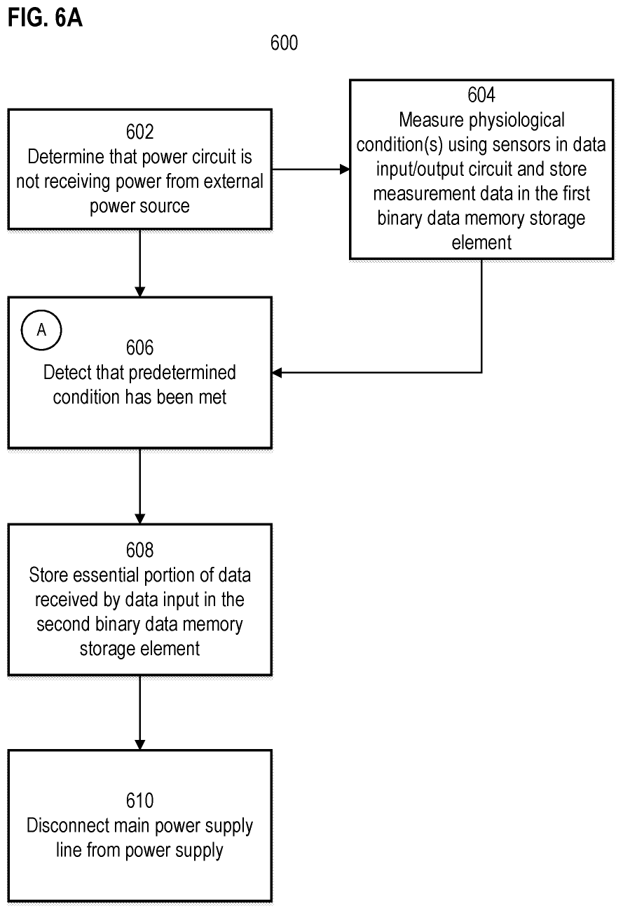

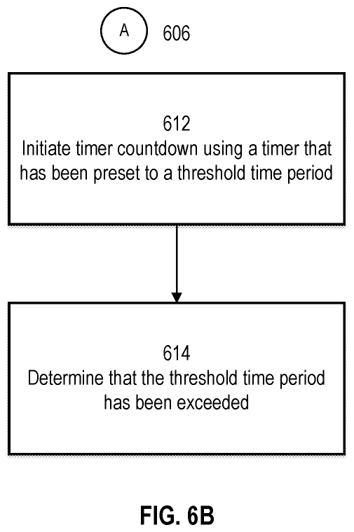

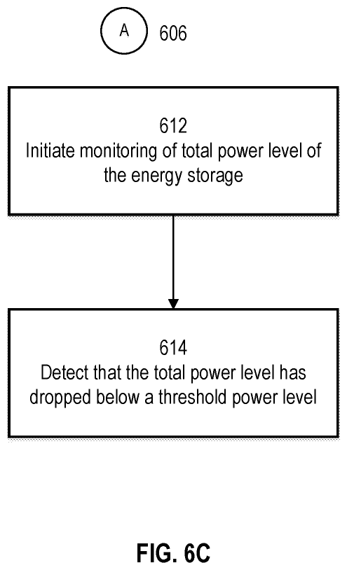

FIG. 6A shows an exemplary method for storing data using an implantable device according to some embodiments of the present disclosure. Specifically, FIG. 6A illustrates a method 600 for allocating an essential portion of data stored in a first element of a memory of the implantable device to a second element of the memory that always receives energy from a power supply of the device. In some embodiments, method 600 may be executed by a digital core of the implantable device (e.g., digital core 204 of implantable device 200 shown in FIG. 2). Prior to the execution of a method for storing data (e.g., method 600), the implantable device may be continuously receiving energy from an external power source. In some embodiments, prior to the execution of a method for storing data (e.g., method 600), all elements of the implantable device may be fully “awake,” i.e., may be electrically coupled to and receiving energy from the power supply of the implantable device.

In some embodiments, the memory of the implantable device may include one or more features of memory 212 of implantable device 200 (shown in FIG. 2). In particular, the first element and the second element of the memory may comprise one or more features of first binary data memory storage element 214 and second binary data memory storage element 216, respectively. In some embodiments, the first element of the memory may be electrically coupled to the energy storage of the device through a main power supply line (e.g., main power supply line 232), while the second element of the memory may be electrically coupled to the energy storage of the device through a separate retention power supply line (e.g., retention power supply line 234). The main power supply line may be continuously supplying energy to the first element of the memory, as well as other elements of the implantable device (e.g., a one or more sensors) prior to the execution of a method for storing data (e.g., method 600) by the digital core. Similarly, the retention power supply line may be continuously supplying energy to the second element of the memory prior to the execution of a method for storing data (e.g., method 600) by the digital core.

In some embodiments, method 600 may begin at a step 602, wherein the digital core may determine that the energy storage in the power management unit of the implantable device is no longer receiving energy from the external power source. In some embodiments, determining that the implantable device is no longer receiving energy from the external power source may comprise receiving one or more signals from the external power source and/or from a user indicating that energy is no longer being provided to the energy storage.

Optionally, after the digital core have determined that the implantable device is no longer receiving energy from the external power source in step 602, method 600 may proceed to a step 604, wherein the digital core may cause one or more sensors of the implantable device to measure one or more physiological signals. Data associated with the measured physiological signals may be transmitted from the one or more sensors to the first element of the implantable device's memory.

In some embodiments, sensing a physiological signal when the implantable device is not receiving energy from the external power source may prevent the external power source from affecting the measurement. For instance, an implantable device may be configured to be implanted in an eye in order to measure an intraocular pressure. This intraocular implant may be configured to receive energy in the form of ultrasonic waves from an interrogator. Specifically, the implant may receive energy from the interrogator when the interrogator is pressed against the eye. The intraocular pressure in the eye may be impacted by the contact between the interrogator and the eye. Only once the interrogator is no longer providing energy to the implant (i.e., once the interrogator is no longer in contact with the eye) can the implant make accurate measurements of the intraocular pressure.

In some embodiments, after the digital core determines that the implantable device is no longer receiving energy from the external power source in step 602 (and, optionally, after physiological signal measurements have been performed in step 604), method 600 may proceed to a step 606, wherein the digital core may detect that a predetermined condition has been met. In some embodiments, the predetermined condition may be associated with an amount of time that has passed since the processors (e.g., digital core) determined that the implantable device is no longer receiving energy from the external power source. In some embodiments, the predetermined condition may be associated with a threshold amount of energy that the energy storage of the implantable device must preserve in order to ensure that essential amounts of data can be stored in the device's memory for an extended period of time. In some embodiments, the predetermined condition may be associated with an amount of data that has been collected.

In some embodiments, after the digital core detects that the predetermined condition has been met in step 606, method 600 may proceed to a step 608, wherein the digital core may cause an essential portion of the data stored in the first element of the implantable device's memory may be transmitted to the second element of the implantable device's memory (e.g., the second binary data memory storage element) that is electrically coupled to the energy storage through the retention power supply line. In some embodiments, the essential portion of the data that is transmitted from the first element of the device's memory to the second element of the device's memory may comprise data associated with a measurement that was performed at a specific moment in time, such as data associated with the most recently performed measurement.

In some embodiments, after the digital core causes an essential portion of the data stored in the first element of the implantable device's memory to be transferred to the second element of the implantable device's memory, method 600 may proceed to a step 610, wherein the processors (e.g., digital core) may disconnect the main power supply line from the energy storage in the device's power management unit. In some embodiments, digital core may disconnect the main power supply line using a power management unit. As mentioned above, the main power supply line may electrically couple the first element of the device's memory to the energy storage. In some embodiments, the main power supply line may electrically couple the sensors or other elements of the implantable device to the energy storage. In some embodiments, disconnecting the main power supply line from the energy storage may prevent the first element of the device's memory (along with other elements connected to the main power supply line) from drawing energy stored in the energy storage. In some embodiments, disconnecting the main power supply line from the power management unit may cause one or more elements of the implantable device to enter a low power “sleep” mode.

The retention power supply line that electrically couples the second element of the device's memory (which stores the essential portion of the data that stored in the first element) may remain connected to the energy storage after the main power supply line has been disconnected in step 610. The second element of the device's memory may be configured to store the essential portion of the data while drawing very little power from the energy storage. In some embodiments, the second element of the device's memory may be configured to store the essential portion of the data using less than or equal to 10, 30, 50, 70, or 100 picowatts of power from the device's energy storage. In some embodiments, the second element of the device's memory may be configured to store the essential portion of the data using greater than or equal to 100, 1000, 5000, 10⁴, or 10⁵ picowatts of power from the device's energy storage. In some embodiments, the second element of the device's memory may be configured to store the essential portion of the device's memory using between 50-150, 100-200, 100-1000, or 100-10⁴ picowatts of power from the device's energy storage. The second element of the device's memory may store the essential portion of the data until the implantable device begins receiving energy from the external power source again.

FIG. 6B shows an exemplary method for detecting that a predetermined condition associated with an implantable device has been met, according to some embodiments of the present disclosure. Specifically, FIG. 6B shows an exemplary method 606 for determining that a threshold amount of time has passed since one or more processors (e.g., digital core) of an implantable device determined that the implantable device is no longer receiving energy from an external power source. Method 606 may be executed during step 606 of method 600 shown in FIG. 6A.

Method 606 may comprise a step 612, wherein a timer countdown may be initiated on a timer that has been pre-set to the threshold time period. The timer may be a component of the digital core. In some embodiments, the countdown may be initiated by the implantable device's digital core after the digital core detects that implantable device is no longer receiving energy from the external power source. In some embodiments, the threshold time period may be less than or equal to 10, 30, 60, 90, or 120 seconds. In some embodiments, the threshold time period may be greater than or equal to 10, 30, 60, 90, or 120 seconds.

Once the countdown completes, method 406 may proceed to step 414, wherein the digital core may determine that the threshold time period has been exceeded. At this point, the digital core may initiate one or more steps intended to put the implantable device into a low power “sleep” mode (e.g., steps 608-610 of method 600 shown in FIG. 6A).

FIG. 6C shows an exemplary method for detecting that a predetermined condition associated with an implantable device has been met, according to some embodiments of the present disclosure. Specifically, FIG. 6C shows a method 606 for determining that a total amount of power stored in a power supply of an implantable device has dropped below a threshold power level. Method 606 may be executed during step 606 of method 600 shown in FIG. 6A.

Method 606 may comprise a step 612, wherein the implantable device's digital core may begin monitoring the total power level of the device's energy storage. In some embodiments, the power level monitoring may be initiated by the implantable device's digital core after the digital core detects that implantable device is no longer receiving energy from the external power source.

In some embodiments, after the power level monitoring is initiated in step 606, method 606 may proceed to step 614, wherein the one or more processors (e.g., the digital core) may determine that the amount of power remaining in the device's power supply has dropped below a threshold power level. At this point, the processors (e.g., the digital core) may initiate one or more steps intended to put the implantable device into a low power “sleep” mode (e.g., steps 608-610 of method 600 shown in FIG. 6A).

In some embodiments, the threshold power level may depend on an operating voltage level of a retention cell in a second element of the device's memory (e.g., retention latch 308 in second binary data memory storage element 304 shown in FIG. 3). For example, an implantable device's energy storage may be a 1 μF capacitor. The device's retention cell may be configured to store data at voltages above 0.4 V and to draw 1 nA of current. The device's processors (e.g., the digital core) may be configured to ensure that the retention cell can store data for a predetermined length of time—for instance, about 26 minutes. Accordingly, the processors (e.g., the digital core) may be configured to cause the device to enter a low power “sleep” mode (i.e., a mode wherein the majority of the device's power is being drawn by the capacitor) whenever the device is not receiving energy from an external power source and the voltage of the capacitor has dropped below 2 V. Note that this example is presented for illustrative purposes only and is not intended to limit the present disclosure.

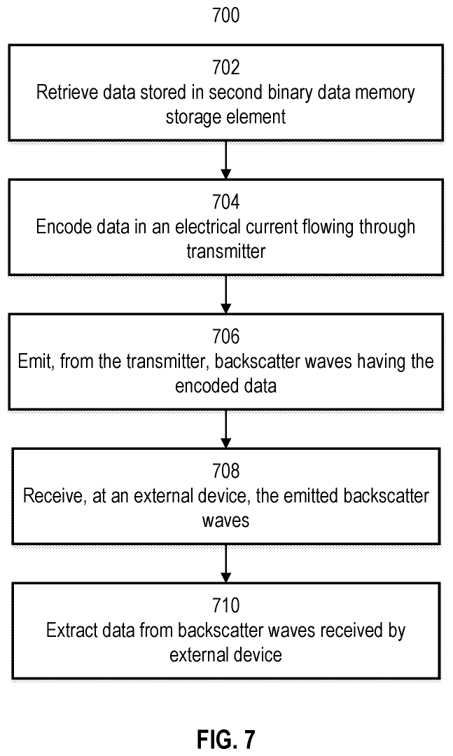

FIG. 7 illustrates an exemplary method for transferring data stored in a memory of an implantable device to an external device. Specifically, FIG. 7 shows a method 700 for transferring data stored in a memory of an implantable device to an external device using backscatter waves. In some embodiments, method 700 may be executed after the implantable device has returned to a full power “awake” mode following a period wherein the implantable device was in a low power “sleep” mode. In such a situation, essential data related to measurements completed before the device entered the low power “sleep” mode may be stored in a second binary data memory storage element of the implantable device's memory (i.e., a binary data memory storage element configured to continuously receive power from the device's energy storage while the device is in “sleep” mode, e.g., second binary data memory storage element 216 shown in FIG. 2).

The implantable device may return to a full power “awake” mode when an external energy source re-initiates energy transfer to the device's power management unit. In some embodiments, once an amount of energy in the device's energy storage has increased beyond a minimum threshold level, the device's processors (e.g., the digital core) may reconnect the device's main power supply line to the power management unit. Once the main power supply line has been reconnected to the power management unit, the device's data input/output circuit, which may receive power through the main power supply line, may begin to function. At this point, method 700 may initiate.

In some embodiments, method 700 may begin at a step 702, wherein the device's digital core may retrieve data stored in the second binary data memory storage element of the device's memory. Method 700 may then proceed to a step 704, wherein a modulation circuit of the implantable device may encode information describing the data in an electrical current flowing through a transmitter (e.g., a transducer) of the implantable device.

After the information describing the data has been encoded in the electrical current flowing through the transmitter in step 704, method 700 may proceed to a step 706, wherein the transmitter may emit backscatter waves having the information encoded in the current. In some embodiments, the backscatter waves may be ultrasonic waves. In some embodiments, the backscatter waves may be radio frequency waves. Method 700 may subsequently proceed to a step 708, wherein the emitted backscatter waves may be received by the external device. Once the emitted backscatter waves are received by the external device, method 700 may proceed to a step 710, wherein the information carried by the backscatter waves may be extracted from the backscatter waves.

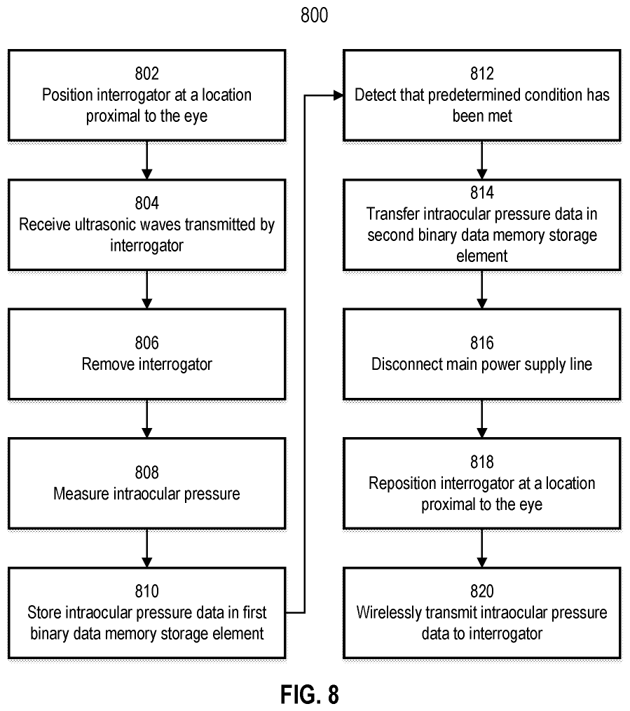

FIG. 8 shows an exemplary method 800 for using an exemplary intraocular implant to measure an intraocular pressure, store intraocular pressure data when the implant is in a low power “sleep” mode, and transmit the intraocular pressure data to an interrogator. Method 800 may allow IOP data to be stored by the ocular implant for extended periods of time before being retrieved by the interrogator without depleting the on-device energy storage.

In some embodiments, method 800 may begin at a step 802, wherein the interrogator may be positioned at a location proximal to the patient's eye in which the ocular implant is implanted. Method 800 may then proceed to a step 804, wherein the ocular implant may receive energy in the form of ultrasonic waves from the interrogator.

After the implantable device has received a sufficient amount of energy from the interrogator, the interrogator may be removed from the location proximal to the patient's eye in a step 806. Method 800 may then proceed to a step 808, wherein an intraocular pressure signal may be measured using one or more pressure sensors in the ocular implant. The one or more sensors may subsequently transmit data associated with the measured IOP to a first binary data memory storage element of the ocular implant's memory in a step 810. The first binary data memory storage element and the one or more sensors may be electrically coupled to the energy storage through a main power supply line. As measurements are made by the sensors and data is stored by the first binary data memory storage element, the amount of energy stored in the energy storage may decrease.

In some embodiments, method 800 may proceed from step 810 to a step 812, wherein a digital core of the ocular implant may detect that a predetermined condition associated with an amount of energy remaining in the energy storage of the ocular implant (see, e.g., exemplary methods 606 shown in FIGS. 4B-4C). Upon detecting that the predetermined condition has been met, method 800 may proceed to a step 814, wherein the digital core may cause a portion of the IOP data stored in the first binary data memory storage element to be transferred to a second binary data memory storage element that is coupled to the energy storage through a retention power supply line. Method 800 may then move to a step 816, wherein the main power supply line may be disconnected from the energy storage, causing the implantable device to enter a low power “sleep” mode.

When a user is ready to retrieve the data stored in the “sleeping” ocular implant, they may execute step 818 by repositioning the interrogator at the location proximal to the patient's eye. Once the interrogator has been repositioned, method 800 may move to a step 820, wherein the IOP data stored in the second binary data memory storage element may be transferred to the interrogator as described in the preceding sections.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

Any of the systems, methods, techniques, and/or features disclosed herein may be combined, in whole or in part, with any other systems, methods, techniques, and/or features disclosed herein.

1. An implantable device comprising:
an energy storage configured to receive power from an external power source;
one or more sensors configured to measure physiological signals;
a memory configured to store physiological signal data measured by the one or more sensor, the memory comprising:
a first binary data memory storage element electrically coupled to the one or more sensors and a main power supply line electrically coupled to the energy storage, and
a second binary data memory storage element electrically coupled to the first binary data memory storage element and a retention power supply line electrically coupled to energy storage; and
a digital circuit configured to:
determine that the power management unit is not receiving power from the external power source;
detect that a predetermined condition has been met;
store an essential portion of the physiological signal data that has been measured by the one or more sensors in the second binary data memory storage element; and
disconnect the main power supply line from the energy storage.

2. The implantable device of claim 1, wherein the main power supply line is configured to transmit a first amount of power from the energy storage to the first binary data memory storage element and the retention power supply line is configured to transmit a second amount of power from the energy storage to the second binary data memory storage element.

3. The implantable device of claim 2, wherein the second amount of power is less than the first amount of power.

4. The implantable device of claim 2 or 3, wherein the second amount of power is less than or equal to 100 picowatts.

5. The implantable device of any one of claims 1-4, wherein the essential portion of the physiological signal data that is stored in the second binary data memory storage element comprises data associated with a most recently performed measurement.

6. The implantable device of any one of claims 1-5, wherein the one or more sensors comprise a pressure sensor.

7. The implantable device of claim 6, wherein the pressure sensor is configured to measure an intraocular pressure.

8. The implantable device of any one of claims 1-7, wherein the one or more sensors comprises one or more electrodes configured to detect an electrophysiological pulse.

9. The implantable device of any one of claims 1-8, wherein the one or more sensors comprises a sensor configured to detect a concentration of an analyte, a pH, a temperature, an evoked action potential in a brain, and/or a local field potential in a brain.

10. The implantable device of any one of claims 1-9, wherein the device is configured to be fully implantable.

11. The implantable device of any one of claims 1-10, wherein the device is configured to be implanted in or attached to a tissue or organ.

12. The implantable device of claim 11, wherein the device is configured to be implanted in an eye.

13. The implantable device of claim 11, wherein the device is configured to be implanted on a on or in a central nervous tissue.

14. The implantable device of claim 11, wherein the device is configured to be implanted on a on or in a brain.

15. The implantable device of claim 11, wherein the device is configured to be implanted on a peripheral nerve.

16. The implantable device of claim 15, wherein the peripheral nerve is a splenic nerve.

17. The implantable device of any one of claims 1-16, wherein the energy storage is configured to receive power wirelessly from the external power source.

18. The implantable device of any one of claims 1-17, wherein the energy storage is configured to receive power from ultrasonic waves produced by the external power source.

19. The implantable device of any one of claims 1-18, wherein the energy storage is configured to receive power from radio frequency waves produced by the external power source.

20. The implantable device of any one of claims 1-19, wherein the energy storage is configured to receive power the external power source via induction.

21. The implantable device of one any of claims 1-20, wherein the energy storage is configured to receive power from the external power source via a capacitive link.

22. The implantable device of any one of claims 1-21, wherein the energy storage is configured to receive power from vibrations produced by the external power source using a vibrational transducer.

23. The implantable device of one any of claims 1-22, wherein the energy storage comprises a capacitor.

24. The implantable device of any one of claims 1-23, wherein the energy storage comprises a battery.

25. The implantable device of any one of claims 1-24, wherein detecting that the predetermined condition has been met comprises determining that a threshold time period from the time that the energy storage last received power from the external device has been exceeded.

26. The implantable device of any one of claims 1-25, wherein detecting that the predetermined condition has been met comprises detecting that a total power level of the energy storage has dropped below a threshold power level.

27. The implantable device of any one of claims 1-26, wherein the first binary data memory storage element comprises a flip-flop circuit.

28. The implantable device of any one of claims 1-27, wherein the second binary data memory storage element comprises a retention latch circuit.

29. A method for collecting and storing data using an implantable device, the implantable device comprising:
an energy storage configured to receive power from an external power source;
one or more sensors configured to measure physiological signals;
a memory configured to store physiological signal data measured by the one or more sensors, the memory comprising:
a first binary data memory storage element electrically coupled to the one or more sensors and a main power supply line electrically coupled to the energy storage, and
a second binary data memory storage element electrically coupled to the first binary data memory storage element and a retention power supply line electrically coupled to the energy storage; and
a digital circuit;
the method comprising:
determining that the energy storage is no longer receiving power from the external power source;
detecting that a predetermined condition has been met;
storing an essential portion of the physiological signal data that has been measured by the one or more sensors in the second binary data memory storage element; and
disconnecting the main power supply line from the energy storage.

30. The method of claim 29, wherein the main power supply line is configured to transmit a first amount of power from the power supply to the first binary data memory storage element and the retention power line is configured to transmit a second amount of power from the power supply to the second binary data memory storage element.

31. The method of claim 30, wherein the second amount of power is less than the first amount of power.

32. The method of claim 30 or 31, wherein the second amount of power is less than or equal to 100 picowatts.

33. The method of any one of claims 29-32, wherein the essential portion of the physiological signal data that is stored in the second binary data memory storage element comprises data associated with a most recently performed measurement.

34. The method of any one of claims 29-33, wherein the one or more sensors comprise a pressure sensor.

35. The method of claim 34, wherein the pressure sensor is configured to measure an intraocular pressure.

36. The method of any one of claims 29-35, wherein the one or more sensors comprise one or more electrodes configured to detect an electrophysiological pulse.

37. The method of any one of claims 29-36, wherein the one or more sensors comprise a sensor configured to detect a concentration of an analyte, a pH, a temperature, and/or an evoked action potential in a brain, and/or a local field potential in a brain.

38. The method of any one of claims 29-37, wherein the device is configured to be fully implantable.

39. The method of any one of claims 29-38, wherein the device is configured to be implanted in or attached to a tissue or organ.

40. The method of claim 39, wherein the device is configured to be implanted in an eye.

41. The method of claim 39, wherein the device is configured to be implanted on or in a central nervous tissue.

42. The method of claim 39, wherein the device is configured to be implanted on a on or in a brain.

43. The method of claim 39, wherein the device is configured to be implanted on a peripheral nerve.

44. The method of claim 43, wherein the peripheral nerve is a splenic nerve.

45. The method of any one of claims 29-44, wherein the energy storage is configured to receive power wirelessly from the external power source.

46. The method of any one of claims 29-45, wherein the energy storage is configured to receive power from ultrasonic waves produced by the external power source.

47. The method of any one of claims 29-46, wherein the energy storage is configured to receive power from radio frequency waves produced by the external power source.

48. The method of any one of claims 29-47, wherein the energy storage is configured to receive power the external power source via induction.

49. The method of one any of claims 29-48, wherein the energy storage is configured to receive power from the external power source via a capacitive link.

50. The method of any one of claims 29-49, wherein the energy storage is configured to receive power from vibrations produced by the external power source using a vibrational transducer.

51. The method of any one of claims 29-50, wherein the energy storage comprises a capacitor.

52. The method of any one of claims 29-51, wherein the energy storage comprises a battery.

53. The method of any one of claims 29-52, wherein detecting that the predetermined condition has been met comprises determining that a threshold time period from the time that the energy storage last received power from the external device has been exceeded.

54. The method of any one of claims 29-53, wherein detecting that the predetermined condition has been met comprises detecting that a total power level of the energy storage has dropped below a threshold power level.

55. The method of any one of claims 29-54, wherein the first binary data memory storage element comprises a flip-flop circuit.

56. The method of any one of claims 29-55, wherein the second binary data memory storage element comprises a retention latch circuit.

57. A non-transitory computer readable storage medium containing instructions for collecting and storing data in an implantable device comprising:
an energy storage configured to receive power from an external power source;
one or more sensors configured to measure physiological signals;
a memory configured to store physiological signal data measured by the one or more sensors, the memory comprising:
a first binary data memory storage element electrically coupled to the one or more sensors and a main power supply line electrically coupled to the energy storage, and
a second binary data memory storage element electrically coupled to the first binary data memory storage element and a retention power supply line electrically coupled to the energy storage;
wherein, when executed by a digital circuit of an electronic device, the instructions cause the electronic device to:
determine that the energy storage is not receiving power from the external power source;
detect that a predetermined condition has been met;
store an essential portion of the physiological signal data that has been measured by the one or more sensors in the second binary data memory storage element; and
disconnect the main power supply line from the energy storage.

58. The non-transitory computer readable storage medium of claim 57, wherein the main power line is configured to transmit a first amount of power from the power supply to the first binary data memory storage element and the retention power line is configured to transmit a second amount of power from the power supply to the second binary data memory storage element.

59. The non-transitory computer readable storage medium of claim 58, wherein the second amount of power is less than the first amount of power.

60. The non-transitory computer readable storage medium of claim 58 or 59, wherein the second amount of power is less than or equal to 100 picowatts.

61. The non-transitory computer readable storage medium of any one of claims 57-60, wherein the essential portion of the data that is stored in the second binary data memory storage element comprises data associated with a most recently performed measurement.

62. The non-transitory computer readable storage medium of any one of claims 57-61, wherein the one or more sensors comprise a pressure sensor.

63. The non-transitory computer readable storage medium of claim 62, wherein the pressure sensor is configured to measure an intraocular pressure.

64. The non-transitory computer readable storage medium of any one of claims 57-63, wherein the one or more sensors comprise one or more electrodes configured to detect an electrophysiological pulse.

65. The non-transitory computer readable storage medium of any one of claims 57-64, wherein the one or more sensors comprises a sensor configured to detect a concentration of an analyte, a pH, a temperature, an evoked action potential in a brain, and/or a local field potential in a brain.

66. The non-transitory computer readable storage medium of any one of claims 57-65, wherein the device is configured to be fully implantable.

67. The non-transitory computer readable storage medium of any one of claims 57-66, wherein the device is configured to be implanted in or attached to a tissue or organ.

68. The non-transitory computer readable storage medium of claim 67, wherein the device is configured to be implanted in an eye.

69. The non-transitory computer readable storage medium of claim 67, wherein the device is configured to be implanted on a on or in a central nervous tissue.

70. The non-transitory computer readable storage medium of claim 67, wherein the device is configured to be implanted on a on or in a brain.

71. The non-transitory computer readable storage medium of claim 67, wherein the device is configured to be implanted on a peripheral nerve.

72. The non-transitory computer readable storage medium of claim 71, wherein the peripheral nerve is a splenic nerve.

73. The non-transitory computer readable storage medium of any one of claims 57-72, wherein the energy storage is configured to receive power wirelessly from the external power source.

74. The non-transitory computer readable storage medium of any one of claims 57-73, wherein the energy storage is configured to receive power from ultrasonic waves produced by the external power source.

75. The non-transitory computer readable storage medium of any one of claims 57-74, wherein the energy storage is configured to receive power from radio frequency waves produced by the external power source.

76. The non-transitory computer readable storage medium of any one of claims 57-75, wherein the energy storage is configured to receive power the external power source via induction.

77. The non-transitory computer readable storage medium of one any of claims 57-76, wherein the energy storage is configured to receive power from the external power source via a capacitive link.

78. The non-transitory computer readable storage medium of any one of claims 57-77, wherein the energy storage is configured to receive power from vibrations produced by the external power source using a vibrational transducer.

79. The non-transitory computer readable storage medium of any one of claims 57-78, wherein the energy storage comprises a capacitor.

80. The non-transitory computer readable storage medium of any one of claims 57-79, wherein the energy storage comprises a battery.

81. The non-transitory computer readable storage medium of any one of claims 57-80, wherein detecting that the predetermined condition has been met comprises determining that a threshold time period from the time that the energy storage last received power from the external device has been exceeded.

82. The non-transitory computer readable storage medium of any one of claims 57-81, wherein detecting that the predetermined condition has been met comprises detecting that a total power level of the energy storage has dropped below a threshold power level.

83. The non-transitory computer readable storage medium of any one of claims 57-82, wherein the first binary data memory storage element comprises a flip-flop circuit.

84. The non-transitory computer readable storage medium of any one of claims 57-83, wherein the second binary data memory storage element comprises a retention latch circuit.
